SEQUENCE LISTING (12 sequences)

SEQ ID NO: 1 - DNA, 20 bp, synthetic construct
  source 1..20: mol_type = other DNA
aagccctccagctcctagtc

SEQ ID NO: 2 - DNA, 20 bp, synthetic construct
  source 1..20: mol_type = other DNA
tccgaagcaccacttcttct

SEQ ID NO: 3 - DNA, 20 bp, synthetic construct
  source 1..20: mol_type = other DNA
gttgaccaacgctggcggga

SEQ ID NO: 4 - DNA, 20 bp, synthetic construct
  source 1..20: mol_type = other DNA
cgggctacctcctccgtcgt

SEQ ID NO: 5 - DNA, 19 bp, synthetic construct
  source 1..19: mol_type = other DNA
ggccaagggtcactacacg

SEQ ID NO: 6 - DNA, 21 bp, synthetic construct
  source 1..21: mol_type = other DNA
gcagtcgcagttttcacactc

SEQ ID NO: 7 - DNA, 20 bp, synthetic construct
  source 1..20: mol_type = other DNA
acatctggctccatgttggg

SEQ ID NO: 8 - DNA, 20 bp, synthetic construct
  source 1..20: mol_type = other DNA
ggcagcatgcaggagtatga

SEQ ID NO: 9 - DNA, 20 bp, synthetic construct
  source 1..20: mol_type = other DNA
tgctgttaaatgtgggggct

SEQ ID NO: 10 - DNA, 20 bp, synthetic construct
  source 1..20: mol_type = other DNA
tcacccttctgaagctgtcg

SEQ ID NO: 11 - DNA, 18 bp, synthetic construct
  source 1..18: mol_type = other DNA
gcaccttcgcgcagttct

SEQ ID NO: 12 - DNA, 18 bp, synthetic construct
  source 1..18: mol_type = other DNA
cccgaactccaccgtgaa

(19) United States
(12) Patent Application Publication
(10) Pub. No.: US 2025/0250538 A1
(43) Pub. Date: Aug. 7, 2025

(54) METHODS FOR MAKING AND USING DIFFERENTIATED NEURAL CELLS

(71) Applicant: RESEARCH DEVELOPMENT FOUNDATION, Carson City, NV (US)

(72) Inventors: Sanae EL HARANE, Geneva (CH); Olivier PREYNAT-SEAUVE, Geneva (CH); Karl-Heinz KRAUSE, Geneva (CH)

(73) Assignee: RESEARCH DEVELOPMENT FOUNDATION, Carson City, NV (US)

(21) Appl. No.: 18/855,867

(22) PCT Filed: Apr. 11, 2023
(86) PCT No.: PCT/US2023/065631

§ 371 Date: Oct. 10, 2024

Related U.S. Application Data
(60) Provisional application No. 63/329,713, filed on Apr. 11, 2022.

(51) Int. Cl. C12N 5/0793; A61K 35/30

(52) U.S. Cl. CPC C12N 5/0619; A61K 35/30; C12N 2500/38; C12N 2501/119; C12N 2501/13; C12N 2501/15; C12N 2501/41; C12N 2501/60; C12N 2501/727; C12N 2501/734; C12N 2506/02; C12N 2513/00

(57) ABSTRACT

The current disclosure provides for methods for differentiating stem and progenitor cells into neural cells through an approach that excludes the use of inhibitors of the BMP4 pathway resulting in SMAD inhibition. Aspects of the disclosure relate to a method for differentiating stem or progenitor cells into neural cells, the method comprising (i) contacting the stem or progenitor cells with a differentiation composition; and (ii) culturing the cells in microwells to form spheroids. Further aspects relate to a method for differentiating stem or progenitor cells into neural cells, the method comprising (i) contacting the stem or progenitor cells with a differentiation composition, wherein the differentiation composition comprises one or more of the ALK inhibitors: DMH1, DMH2, K02288, A83-01, or combinations thereof; and (ii) culturing the cells in microwells to form spheroids. Also described is a neural cell, spheroid, a population of cells, or a population of spheroids produced by the methods of the claims.

### CROSS-REFERENCE TO RELATED APPLICATIONS

This application claims the benefit of priority to U.S. Provisional Patent Application Ser. No. 63/329,713, filed Apr. 11, 2022, hereby incorporated by reference in its entirety.

### SEQUENCE LISTING

The instant application contains a Sequence Listing which has been submitted in XML format and is hereby incorporated by reference in its entirety. Said XML copy, created on Apr. 11, 2023, is named “CLFRPO404WO_SL” and is 12 kilobytes in size.

### BACKGROUND OF THE INVENTION

### I. Field of the Invention

This invention relates to the field of cell biology and treatment of disease.

### II. Background

Cell populations that retain the ability to differentiate into numerous specialized cell types are useful for developing large numbers of lineage specific differentiated cell populations. These lineage specific differentiated cell populations are contemplated to find use in cell replacement therapies for patients with diseases resulting in loss of function of a defined cell population. In addition to their direct therapeutic value, lineage specific differentiated cells are also valuable research tools for a variety of purposes including in vitro screening assays to identify, confirm, and test for specification of function or for testing delivery of therapeutic molecules to treat cell lineage specific disease. In the case of Parkinson's disease, for example, it is the loss of midbrain dopaminergic (DA) neurons that results in the appearance of disease symptoms. Thus, there is need for methods of producing DA neuronal cells from pluripotent cells, since such cells could be used both therapeutically and in disease models, e.g., to identify new therapeutics for treatments for neurodegenerative disease.

### SUMMARY OF THE INVENTION

The current disclosure provides for methods for differentiating stem and progenitor cells into neural cells through an approach that excludes the use of inhibitors of the BMP4 pathway resulting in SMAD inhibition. The method include a method for differentiating stem or progenitor cells into neural cells, the method comprising (i) contacting the stem or progenitor cells with a differentiation composition; and (ii) culturing the cells in microwells to form spheroids and/or neurospheres. Also described is a method for differentiating stem or progenitor cells into neural cells, the method comprising (i) contacting the stem or progenitor cells with a differentiation composition, wherein the differentiation composition comprises one or more of the ALK inhibitor(s): DMH1, DMH2, K02288, and A83-01; and (ii) culturing the cells in microwells to form spheroids and/or neurospheres. Also described is a neural cell, spheroid, a population of cells, or a population of spheroids produced by the methods of the claims. Methods also include a method of treating a disease in a mammalian subject comprising administering to the subject a therapeutically effective number of neural cells, spheroids, or neurospheres, a population of cells, spheroids, or neurospheres of the disclosure. Further described is a method of screening a test compound comprising: (a) contacting the test compound with cells, spheroids, or neurospheres of the disclosure; and (b) measuring the function, physiology, or viability of the cells.

The term spheroid refers to cells in a three-dimensional spherical configuration. The spheroid may be comprised of progenitor cells or substantially of progenitor cells such as or such as at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) of the cells in the spheroid may be progenitor. A neurosphere is comprises of cells that are further differentiated, such as cells that are committed to neural cell lineages. A neurosphere may comprise or comprise at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) cells that are neural progenitors.

The compounds, DMH1, DMH2, K02288, and A83-01, are also referred to as ALK inhibitors. The ALK inhibitor may consist of DMH1, DMH2, K02288, or A83-01. The ALK inhibitors may comprise or consist of DMH2. The ALK inhibitors may consist of DMH1 and DMH2. The ALK inhibitors may consist of K02288 and DMH2. The ALK inhibitors may consist of A8301 and DMH2.

The stem or progenitor cells may comprise induced pluripotent stem cells (iPSCs) or embryonic stem (ES) cells. The stem or progenitor cells may comprise hematopoietic stem or progenitor cells. The stem or progenitor cells may be further defined as totipotent, pluripotent, or multipotent stem cells. The cells may comprise embryonic stem (ES) cells. The cells may be human cells or are derived from human cells. The cells may be human ES cells. The human ES cells may comprise or be further defined as HS420 cells. The stem or progenitor cells may exclude iPSCs, ES cells, hematopoietic stem or progenitor cells, totipotent cells, pluripotent cells, multipotent stem cells, human cells, cells derived from human cells, or HS420 cells.

Contacting the cells may comprise contacting the cells for a time period of about 1-7 days of substantially continuous contact. The term “substantially continuous contact” refers to a contact that is for at least 90, 91, 92, 93, 94, 95, 96, 97, 98, 99, or 100% of the time during a certain time period, but does not exclude brief periods of non-contact, such as periods in which the cells may be undergoing a washing, re-plating, trypsinization, or a change in the cell culture medium. The time period, such as the time period a compound or ALK inhibitor is contacted with the cells, may comprise at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days (or any derivable range therein). The contacting, such as the contacting of cells with a compound or ALK inhibitor described herein, may be defined from a certain time period, such as from when the differentiation medium is added (FIG. 12), wherein Day 0 is the day that the cells are first contacted with the differentiation medium. The contacting may begin at day −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 and end at a specific day, such as day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The cells may be contacted with 0.01-5 μM of a compound described herein and/or ALK inhibitor. The cells may be contacted with at least, at most, about, or exactly 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, or 20 μM (or any derivable range therein) of a compound or ALK inhibitor described herein. The cells may be contacted with 0.2 μM of a compound or ALK inhibitor described herein. The cells may be contacted with 0.8 μM of a compound or ALK inhibitor described herein.

The method may further comprise contacting the cells with a Rho Kinase (ROCK) inhibitor. The methods may exclude contacting the cells with a ROCK inhibitor. The ROCK inhibitor may comprise Y27632. Other ROCK inhibitors useful in the methods of the disclosure include Fasudil, Ripasudil, Netarsudil, RKI-1447, GSK429286A, and Y30141. The methods may exclude contacting the cells with Y27632, Fasudil, Ripasudil, Netarsudil, RKI-1447, GSK429286A, and/or Y30141. The cells may be contacted with 5-15 μM ROCK inhibitor. The cells may be contacted with at least, at most, about, or exactly 1, 1.5, 2, 2.5, 3, 3.5, 4, 4.5, 5, 5.5, 6, 6.5, 7, 7.5, 8, 9, 10, 11, 12, 13, 14, 15, 16, 17, 18, 19, 20, 21, 22, 23, 24, 25, 30, 35, 40, 45, 50, 55, 60, 65, 70, 75, or 80 μM, nM, or mM ROCK inhibitor (or any derivable range therein). The cells may be contacted with the ROCK inhibitor prior to contact with the ALK inhibitor. The cells may be contacted with the ROCK inhibitor immediately prior to contact with the ALK inhibitor. The contact with the ROCK inhibitor and the contact with the ALK inhibitor compound may be overlapping for a time period. The contact with the ROCK inhibitor and the contact with the ALK inhibitor may be non-overlapping and comprises a time period between when the cells are contacted with the ROCK inhibitor and when the cells are contacted with the ALK inhibitor. The cells may be contacted with the ROCK inhibitor after the ALK inhibitor. The contact with the ROCK inhibitor and the contact with the ALK inhibitor may be non-overlapping and comprises a time period between when the cells are contacted with the ALK inhibitor and when the cells are contacted with the ROCK inhibitor. The time period may be at least, at most, or about 1, 2, 3, 4, 5, 10, 15, 30, or 45 min or 1, 2, 3, 4, 5, 6, 7, 8, 9, 10, 12, 18, or 24 hours, or 1, 2, 3, 4, 5, 6, 7, 8, 9, or 10 days (or any derivable range therein). The cells may be contacted with the ROCK inhibitor for a time period of 1-48 hours. The cells may be contacted with the ROCK inhibitor for a time period of 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours, or 2.5, 3, 3.5, 4, 4.5, 5, 5.5, 6, 6.5, or 7 days, or 1, 2, 3, 4, or 5 weeks (or any derivable range therein). The contacting with the ROCK inhibitor may be defined from a certain time period, such as from when the differentiation medium is added (FIG. 12), wherein Day 0 is the day that the cells are first contacted with the differentiation medium. The contacting may begin at day −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 and end at a specific day, such as day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The method may exclude contacting the cells, spheroids, or neurospheres with one or more Smad inhibitors. The method may exclude contacting the cells, spheroids, or neurospheres with LDN193189 and/or SB431542. The method may exclude contacting the cells, spheroids, or neurospheres with LDN193189 and SB431542. The method may exclude dual or mono-Smad inhibition. The method may exclude contacting the cells with a Noggin protein or Noggin modulator, such as an activator or repressor of Noggin or a direct activator or repressor of Noggin. The method may exclude contacting the cells with a BMP4 inhibitor. The method may exclude contacting the cells with Noggin and/or Chordin or activators thereof.

The neural cells may be further defined as dopaminergic neurons, glutamatergic, serotoninergic, cholinergic, GABAergic, motoneurons, astrocytes, or oligodendrocytes. The neural cells may be further defined as a neural cell described herein. The term “dopaminergic neuron” or “DA neuron” refers to a neuron having an ability to produce dopamine (3,4-dihydroxyphenylethylamine). A dopaminergic neuron does not need to produce dopamine all the time, but only needs to have dopamine production capability. The DA neuron may be a DA neuron of the A8 group, A9 group, A10 group, A11 group, A12 group, A13 group, A14 group, A15 group, A16 group, Aaq group, or telencephalic group. The neural cells may exclude dopaminergic neurons, glutamatergic, serotoninergic, cholinergic, GABAergic, motoneurons, astrocytes, or oligodendrocytes. The DA neuron may exclude a DA neuron of the A8 group, A9 group, A10 group, A11 group, A12 group, A13 group, A14 group, A15 group, A16 group, Aaq group, or telencephalic group.

Contacting the cells with a compound or ALK inhibitor described herein may comprise culturing the cells in a cell culture medium comprising the compound or ALK inhibitor. The cell culture medium may comprise one or more of DMEM medium, Neurobasal medium, a GSK inhibitor, cAMP, GDNF, BDNF, amino acids, X-VIVO medium, antimicrobial agents, B-27 supplement, N-2 supplement and L-glutamin, sonic hedgehog protein (SHH), purmorphamin, FGF-8 protein (fibroblast growth factor 8), FGF-20, TGF-B3, and a gamma secretase inhibitor. Gamma secretase inhibitors include, for example DAPT (ref CAS 208255-80-5) and Compound E (ref CAS 209986-17-4). The methods may exclude contacting the cells with one or more of DMEM medium, Neurobasal medium, a GSK inhibitor, cAMP, GDNF, BDNF, amino acids, X-VIVO medium, antimicrobial agents, B-27 supplement, N-2 supplement and L-glutamin, sonic hedgehog protein (SHH), purmorphamin, FGF-8 protein (fibroblast growth factor 8), FGF-20, TGF-B3, and a gamma secretase inhibitor, DAPT (ref CAS 208255-80-5), Compound E (ref CAS 209986-17-4), a GSK3 inhibitor, or CHIR99021. The cells may be further contacted with a GSK3 (glycogen synthase kinase 3) inhibitor for a period of time. The GSK3 inhibitor may comprise CHIR99021. The GSK3 inhibitor may be added after 3 days after contact with the differentiation medium. The GSK3 inhibitor may be added 2, 3, 4, 5, 6, 7, or 8 days (or any derivable range therein) after contact with the differentiation medium. The time period may be 5-15 days. The time period may be 10 days. The contacting with the GSK3 inhibitor, cAMP, GDNF, BDNF, amino acids, X-VIVO medium, antimicrobial agents, B-27 supplement, N-2 supplement, L-glutamin, sonic hedgehog protein (SHH), purmorphamin, FGF-8 protein (fibroblast growth factor 8), FGF-20, TGF-B3, gamma secretase inhibitor, DAPT (ref CAS 208255-80-5), Compound E (ref CAS 209986-17-4), and/or CHIR99021 may begin at day −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 and end at a specific day, such as day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The method may comprise or further comprise contacting the cells with one or more of FHF8, SHH, and purmorphamin for a period of time. The cells may be contacted with FHF8, SHH, and/or purmorphamin one day after contact with the differentiation medium. The period of time may be 3-10 days. The period of time may be 7 days.

The method may comprise or further comprise contacting the cells with one or more of cAMP, GDNF, BDNF, TGFB3, FGF20, and a gamma secretase inhibitor for a period of time. The cells may be contacted with cAMP, GDNF, BDNF, TGFB3, FGF20, and/or a gamma secretase inhibitor eight days after contact with the differentiation medium. The period of time may be 15-40 days.

The contacting with the GSK3 inhibitor, cAMP, GDNF, BDNF, amino acids, X-VIVO medium, antimicrobial agents, B-27 supplement, N-2 supplement, L-glutamin, sonic hedgehog protein (SHH), purmorphamin, FGF-8 protein (fibroblast growth factor 8), FGF-0, TGFB3, gamma secretase inhibitor, DAPT (ref CAS 208255-80-5), Compound E (ref CAS 209986-17-4), and/or CHIR99021 may begin at day −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 and end at a specific day, such as day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The method may comprise or further comprise contacting the cells with ascorbic acid. The ascorbic acid may be at a concentration of 100-400 μM. The concentration of the ascorbic acid may be, be at least, or be at most, 20, 25, 30, 35, 40, 45, 50, 55, 60, 65, 70, 75, 80, 85, 90, 95, 100, 105, 110,115, 120, 125, 130, 135, 140,145, 150, 155, 160,165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, or 700 μM (or any derivable range therein). The ascorbic acid may be in a concentration of 200 μM when in contact with the cells. The cells may be contacted with ascorbic acid at a period of time of 8-15 days after contact with the differentiation medium. The cells may be contacted with the ascorbic acid at a time period of, or at least, or of at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days (or any derivable range therein) after contact with the differentiation medium. The methods may exclude contacting the cells with ascorbic acid. The cells may be contacted with the ascorbic acid at a period of time of 13 days after contact with the differentiation medium. The cells may be contacted with the ascorbic acid for a time period of 10-40 days. The cells may be contacted with the ascorbic acid for a time period of, of at least, or of at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days, or any derivable range therein. The contacting with ascorbic acid may begin at day −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 and end at a specific day, such as day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The differentiation composition may comprise or the method may further comprise contacting the cells, spheroids, or neurospheres with an extracellular matrix for a period of time. The extracellular matrix may be Laminin and/or Geltrex. The methods may exclude contacting the cells, spheroids, or neurospheres with an extracellular matrix. The differentiation composition may exclude an extracellular matrix. Other extracellular matrices that may be included or excluded in the methods and compositions include collagen, elastin, fribronectin, hyaluronic acid, tenascins, hyaluoronans, and proteoglycans. The differentiation composition may comprise or the method may further comprise contacting the cells, spheroids, or neurospheres with RGD peptides for a period of time. The tripeptide Arg-Gly-Asp (RGD) consists of Arginine, Glycine, and Aspartate. It was originally identified as the amino acid sequence within the extracellular matrix protein fibronectin that mediates cell attachment.

The cells, spheroids, and/or neurospheres may be cultured under hypoxic conditions for a period of time. The period of time may be from 1 to 60 days. The hypoxic conditions may comprise 0-10% oxygen. The hypoxic condition may be 2-5% oxygen or may be 3% oxygen. The period of time may be from 1 to 60 days. The hypoxic conditions may comprise, comprise at least, or comprise at most 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% oxygen, or any derivable range therein. The time period may be 1, 2, 3, 4, 5, 6, 7, 8, 9, 10, 11, 12, 13, 14, 15, 16, 17, 18, 19, or 20 days, or any derivable range therein. The hypoxic condition may begin at day −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 and end at a specific day, such as day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The methods may comprise or further comprise contacting the cells with a HIF-1α stabilizer. The HIF-1α stabilizer may comprise one or more of dimethyloxalyl glycine, FG4592, CoCl₂, Deferoxamine mesylate, cyclometalated iridium(III) metal complex 1a, 1-(Imidazol-1-ylmethyl)-3,5-diphenylpyrazole, 3,5-Diphenyl-1-(pyrazole-1-ylmethyl) pyrazole, N-[(3,5-diphenylpyrazol-1-yl) methyl]-N-phenylaniline, (3,5-Diphenylpyrazol-1-yl) methyl]diethylamine, and (3,5-Diphenylpyrazol-1-yl) methyl] diisopropylamine. The methods may exclude contacting the cells with a HIF-1α stabilizer. The methods may exclude contacting the cells with dimethyloxalyl glycine, FG4592, CoCl₂, Deferoxamine mesylate, cyclometalated iridium(III) metal complex 1a, 1-(Imidazol-1-ylmethyl)-3,5-diphenylpyrazole, 3,5-Diphenyl-1-(pyrazole-1-ylmethyl) pyrazole, N-[(3,5-diphenylpyrazol-1-yl) methyl]-N-phenylaniline, (3,5-Diphenylpyrazol-1-yl) methyl]diethylamine, and/or (3,5-Diphenylpyrazol-1-yl) methyl] diisopropylamine. The HIF-1α stabilizer may be contacted with the cells for a time period. The time period may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 days, or any derivable range therein. The HIF-1α stabilizer contact with the cells may begin at day −14, −13, −12, −11, −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 and end at a specific day, such as day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The ratio of the number of spheroids or neurospheres to the volume of cell culture media may be about 1000 spheroids or neurospheres to 200-1000 μl cell culture media. The ratio of the number of spheroids or neurospheres to the volume of cell culture media may be about 1000 spheroids or neurospheres to 200-500 μl cell culture media. The ratio of the number of spheroids or neurospheres to the volume of cell culture media may be about, may be at most, or may be at least 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 spheroids or neurospheres to about, to at least, or to at most 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1000 μl cell culture media or any derivable range therein.

The ratio may be maintained for at least a time period of Day 0 to Day 42, wherein Day 0 is the day that the cells are first contacted with the differentiation medium. The ratio may be maintained at a time period of, of at least, or of at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any derivable range therein.

The stem or progenitor cells may comprise exogenously expressed Nurr-1 and/or Pitx3. The stem or progenitor cells may comprise a heterologous nucleic acid encoding for a Nurr-1 and/or Pitx3 protein or a functional fragment thereof. The neural cells may be further defined as Nestin+, Pax-6+, and Sox-1+ cells. The methods may exclude cells comprising or the cells may exclude exogenous expression of and/or a heterologous nucleic acid encoding for Nurr-1 and/or Pitx3.

Culturing the cells in microwells may comprise culturing the cells on a substrate material comprising a hydrophilic and porous material layer with a plurality of lasting well-shaped indents on its outer surface supported by a semipermeable membrane on its inner surface, wherein said well-shaped indents have an aperture from 100 pm² to about 1 mm² and a bottom surface from about 100 pm² to about 1 mm², wherein said hydrophilic and porous material layer has a porosity which allows the passage of oxygen and cell nutrients. The semipermeable membrane may have pores having a diameter from about 1 μm to about 200 nm, for example from about 2 μm to about 40 nm. The semipermeable membrane may have pores having a diameter of, of at least, or of most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 (or any derivable range therein) pm or nm.

The percentage of non-neural cells in the cell culture, spheroid, or neurosphere after contact with a compound or ALK inhibitor described herein for a period of time may be less than 30% in the population of cells of the current disclosure. The percentage of non-neural cells in the cell culture after contact with a compound or ALK inhibitor described herein for a period of time may be less than 40%, 35%, 30%, 25%, 20%, 15%, 10%, 9%, 8%, 7%, 6%, 5%, 4%, 3%, 2%, or 1% in the population of cells (or any derivable range therein). The period of time may be 4-8 days. The time period may be at least, at most, or about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days, or any derivable range therein.

The term “3D cell culture” as used herein refers to cell culture wherein cells are permitted to grow or interact with their surroundings in all three dimensions. This can be achieved by growing the cells on low adherence plates, bioreactors, or small capsules. The cells grown in 3D culture may take the shape of a spheroid or neurosphere as they grow.

The term “cells” as used herein may include spheroids and neurospheres, since these are made up of cells, unless it is stated otherwise.

The methods may exclude contacting the cells with an ALK inhibitor, DMH1, DMH2, K02288, A83-01, a ROCK inhibitor, Y27632, Fasudil, Ripasudil, Netarsudil, RKI-1447, GSK429286A, Y30141, LDN193189, SB431542, DMEM medium, Neurobasal medium, a GSK inhibitor, cAMP, GDNF, BDNF, amino acids, X-VIVO medium, antimicrobial agents, B-27 supplement, N-2 supplement and L-glutamin, SHH, purmorphamin, FGF-8 protein, FGF-20, TGF-B3, and a gamma secretase inhibitor, DAPT (ref CAS 208255-80-5), Compound E (ref CAS 209986-17-4), a GSK3 inhibitor, CHIR99021, dimethyloxalyl glycine, FG4592, CoCl₂, Deferoxamine mesylate, cyclometalated iridium(III) metal complex 1a, 1-(Imidazol-1-ylmethyl)-3,5-diphenylpyrazole, 3,5-Diphenyl-1-(pyrazole-1-ylmethyl) pyrazole, N-[(3,5-diphenylpyrazol-1-yl) methyl]-N-phenylaniline, (3,5-Diphenylpyrazol-1-yl) methyl]diethylamine, and/or (3,5-Diphenylpyrazol-1-yl) methyl] diisopropylamine at day −14−, −13−, −12−, −11−, −10−, −9−, −8−, −7−, −6−, −5−, −4−, −3−, −2−, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100, or any derivable range therein.

The disease may comprise a neurodegenerative disease. The subject may be a human subject. The subject may be a mammal. The subject may comprise a laboratory animal, pig, rat, goat, rabbit, cat, dog, horse, or mouse. The spheroids or neurospheres may be dissociated prior to administration. The method may exclude dissociation of the spheroids or neurospheres prior to administration.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1B. Dopaminergic differentiation experiment. Shown are culture methods including 3D-immersion (A) and 3D-AirLiwell (B)

FIG. 2. Dopaminergic neurospheres are more homogenous and standardized.

FIG. 3. Neural precursors and neuronal markers expression by qPCR (top) and Midbrain & dopaminergic specific markers expression by qPCR (bottom). FIG. 3 shows that there is better neural induction and specification for some markers for 3D-ALi neurospheres.

FIG. 4. Gene expression fold-change 3D-ALi/3D-i after 6 weeks of differentiation by RNA-seq. FIG. 4 shows that there is higher expression of dopaminergic precursors markers by 3D-ALi but a lack of neuronal maturation.

FIG. 5. Gene expression fold-change 3D-ALi/3D-i after 6 weeks of differentiation by RNA-seq. After 6 weeks of culture the neurospheres cultivated by 3D-ALi shown a down-regulation of some cell proliferation markers.

FIG. 6. Tyrosine hydroxylase expression by western blot. 3D-ALi have the ability to produce dopamine but present a lack of maturation confirmed by western blot & HPLC.

FIG. 7. Dopamine dosage by HPLC. 3D-ALi have the ability to produce dopamine but present a lack of maturation confirmed by western blot & HPLC.

FIG. 8. 3D-Ali neurospheres are electrophysiologically functional and have an electrical signal more stable and homogenous.

FIG. 9. 3D-Ali dopaminergic neurospheres cultivated on extracellular matrix have the ability to mature.

FIG. 10. 3D-Ali dopaminergic neurons observed by electron microscopy are more homogenous and pure.

FIG. 11 shows the chemical structures of DMH1, DMH2, A83-01, and K02288.

FIG. 12A-B exemplifies method aspects for differentiation of stem cells into neural cells using 3D culture.

FIG. 13A-C. Single-cell RNA-seq analysis of dopaminergic neurospheres at 6 weeks.

FIG. 14. Quantitative PCR analysis of dopaminergic neurospheres at 6 weeks.

FIG. 15. 3D-immersion neurospheres produce more dopamine than 3D-Ali neurospheres as measured by HPLC.

FIG. 16. Electrical signal of neurons contained in 3D-Ali neurospheres is more similar to the physiology and is highly standardized.

FIG. 17. Experimental design and engineering of maturation protocol.

FIG. 18. Effect of hypoxia and laminin on dopaminergic maturation.

FIG. 19. Testing of the volume:neurosphere ratio and its effects on dopaminergic maturation.

FIG. 20. Protocol design with the addition of anti-oxidants.

FIG. 21A-B. Effects of DA maturation with the additional of anti-oxidants. FIG. 21A shows the overall morphology of the neurospheres with the addition of anti-oxidants. FIG. 21B shows the impact of certain markers when the neurospheres are cultured with the anti-oxidants in a 3D-Ali method.

FIG. 22. Dopaminergic differentiation adding Alk-I compounds in 3D-Ali protocol. The concentrations of the compounds tested were 0.2 μM and 0.8 μM.

FIG. 23A-D. Quantitative PCR results: Dopaminergic differentiation adding Alk-I compounds in 3D-Ali protocol. Shown are qPCR results at week 2 and week 4 for (A) Ki-67 and Nestin; (B) Pax6 and B3-Tub; (C) Lmx1a and Nurr-1; and (D) Map-2 and TH. The data in FIGS. 23A-D shown in each group of 12 bars represents, from left to right: 1: Control day 0, 2: LDN193189/SB431542, 3: DMSO 0.2 μM, 4: DMSO 0.8 μM, 5: DMH1 0.2 μM, 6: DMH2 0.2 μM, 7: A-8301 0.2 μM, 8: K02288 0.2 μM, 9: DMH1 0.8 μM, 10: DMH2 0.8 μM, 11: A-8301 0.8 μM, 12: K02288 0.8 μM. The 11 bars in the bottom bar graph in FIG. 23D represent data, from left to right, from: 1: Control day 0, 2: LDN193189/SB431542, 3: DMSO 0.2 μM, 4: DMSO 0.8 μM, 5: DMH1 0.2 μM, 6: DMH2 0.2 μM, 7: A-8301 0.2 μM, 8: K02288 0.2 μM, 9: DMH1 0.8 μM, 10: DMH2 0.8 μM, and 11: A-8301 0.8 μM.

### DETAILED DESCRIPTION OF THE INVENTION

Formation of neuroectoderm is a crucial step in the differentiation of pluripotent stem cells towards neural cells and tissues. The current disclosure provides an effective alternative to methods that use a Smad inhibition protocol (Noggin, LDN193189, and SB431542 in various combinations). The inventors hypothesized that differentiating progenitor cells into mature neurons in a three-dimensional cell culture would provide advantages over differentiation methods in a two-dimensional cell culture. These advantages include: 1) no cell detachment (which may induce chromosomic alterations and increase cancerous risk of cells); 2) reduction in dell mortality due to enzymatic cell detachment; 3) simplification of cell culture protocols; 4) improved standardization and homogeneity of transplanted cells; 5) cells are more representative of physiological neural tissue; and 6) improved yield of neuronal differentiated cells.

### I. Definitions

The term “differentiation” as used with respect to cells in a differentiating cell system refers to the process by which cells differentiate from one cell type (e.g., a multipotent, totipotent or pluripotent differentiable cell) to another cell type such as a fully differentiated cell. More generally, the term “differentiation” refers to a process whereby an unspecialized stem cell or a precursor cell acquires the features of a specialized or fully differentiated cell such as a brain, heart, liver, or muscle cell. Differentiation is controlled by the interaction of a cell's genes with the physical and chemical conditions outside the cell, usually through signaling pathways involving proteins embedded in the cell surface.

Progenitor cells in this disclosure are included within somatic cells. Progenitor cells are multipotent cells. Totipotent, pluripotent, and multipotent cells can be “stem cells,” which are capable of differentiating into one or more different cell types. The term “stem cells,” “embryonic stem cell”, “induced pluripotent stem cell” have been described above.

In the present specification, the “stem cell” refers to a cell that can be cultured in vitro and can be differentiated into cells of plural lineages constituting the body. It specifically includes ES cell, pluripotent stem cell derived from fetal primordial germ cell (EG cell: Proc Natl Acad Sci USA. 1998, 95: 13726-31), pluripotent stem cell derived from testis (GS cell: Nature. 2008, 456: 344-9), induced pluripotent stem cell derived from somatic cell (induced pluripotent stem cells; iPS cell), and human pluripotent somatic stem cell (neural stem cell), preferably iPS cell and ES cell, more preferably iPS cell.

### II. ALK Inhibitors

Pluripotent Stem cells (PSCs) derived from embryos or induced from somatic cells have the capacity to differentiate into a wide variety of cell types of interest for tissue modelling and cell therapy. Mimicking embryonic development in vitro provide the best approach for generating differentiated cells with defined properties. The first natural specification of embryonic tissues occurs at the gastrulation stage, with the differentiation of the three germ layers ectoderm, mesoderm and endoderm from which all the adult tissues will derive.

Multiple combinations between Noggin, LDN 193189 and SB 431542 are commonly used and generally called “dualSMAD inhibition”. It represents to date the most widely used way to induce early neural specification of PSC in vitro. Described herein is the report of a differentiation approach that excludes “dual Smad inhibition,” and instead achieves differentiation through the use of 4 chemicals inhibiting ALK to induce neural specification of pluripotent stem cells in vitro: DMH1, DMH2, K02288 and A83-01 (Table 1).

TABLE 1
Compounds

| | Alternative | | | MW |
|---|---|---|---|---|
| Name | Name | Chemical Name | Formula | g/mol |
| DMH1 | Dorsomorphin | 4-[6-[4-(1- | C₂₄H₂₀N₄O | 380.44 |
| | homolog 1 | Methylethoxy)phenyl]pyrazolo[1,5- | | |
| | | a]pyrimidin-3-yl]-quinoline | | |
| DMH2 | | 4-[6-[4-[2-(4- | C₂₇H₂₅N₅O₂ | 451.52 |
| | | Morpholinyl)ethoxy]phenyl]pyrazolo[1,5- | | |
| | | a]pyrimidin-3-yl]quinoline | | |
| A83-01 | A-83-01 | 3-(6-Methyl-2-pyridinyl)-N -phenyl-4-(4- | C₂₅H₁₉N₅S | 421.52 |
| | | quinolinyl)-1H -pyrazole-1-carbothioamide | | |
| K02288 | K02288a | 3-[(6-Amino-5-(3,4,5-trimethoxyphenyl)-3- | C₂₀H₂₀N₂O₄ | 352.38 |
| | | pyridinyl]phenol | | |

### III. Cell Sources

The disclosure relates to the differentiation of a starting population of stem or progenitor cells into neural cells. The stem or progenitor cells may be one described herein and/or derived from a source described herein. The stem or progenitor cell may be an ES cell. It is contemplated that an ES cell derived from any warm-blooded animal, preferably mammal can be used. Examples of the mammal include mouse, rat, guinea pig, hamster, rabbit, cat, dog, sheep, swine, bovine, horse, goat, monkey, and human. Preferable examples of the ES cell include ES cells derived from human. The stem or progenitor cell may exclude a cell derived from or taken from a human fetus.

Specific examples of the ES cell include an ES cell of a mammal and the like, which has been established by culturing an early embryo prior to implantation, an ES cell established by culturing an early embryo prepared by nucleus transplantation of the nucleus of a somatic cell, and an ES cell obtained by alteration of a gene on the chromosomes of these ES cells by a genetic engineering method. Each ES cell can be prepared according to a method generally performed in the pertinent field, or a known document.

Mouse ES cell was established in 1981 by Evans et al (1981, Nature 292: 154-6) and Martin G R. et al. (1981, Proc Natl Acad Sci 78: 7634-8) and can be purchased from, for example, Sumitomo Dainippon Pharma Co., Ltd. (Osaka, Japan) and the like.

Human ES cell was established in 1998 by Thomson et al (Science, 1998, 282: 1145-7), and is available from WiCell Research Institute (available on the world wide web at wicell.org/, Madison, Wis., USA), US National Institute of Health, Kyoto University and the like and can be purchased from, for example, Cellartis (available on the world wide web at cellartis.com/, Sweden) and the like.

The stem or progenitor cell may be an iPSC (also known as iPS cell). As an iPS cell, an iPS cell derived from any warm-blooded animal, preferably mammal, can be used. Examples of the mammal include mouse, rat, guinea pig, hamster, rabbit, cat, dog, sheep, swine, bovine, horse, goat, monkey, and human. Preferable examples of the iPS cell include an iPS cell derived from human.

Specific examples of the iPS cell include a cell that acquired multipotency as in ES cell, which can be obtained by introducing plural genes into a somatic cell such as skin cell and the like. For example, an iPS cell obtained by introducing Oct3/4 gene, Klf4 gene, c-Myc gene and Sox2 gene, and an iPS cell obtained by introducing Oct3/4 gene, Klf4 gene and Sox2 gene (Nat Biotechnol 2008; 26: 101-106). Other than these, a method of further decreasing transgene (Nature. 2008 Jul. 31; 454 (7204): 646-50), a method utilizing a low-molecular-weight compound (Cell Stem Cell. 2009 Jan. 9; 4(1): 16-9, Cell Stem Cell. 2009 Nov. 6; 5(5): 491-503), a method utilizing a transcription factor protein instead of gene (Cell Stem Cell. 2009 May 8; 4(5): 381-4) and the like. The produced iPS cell can be used for the present invention irrespective of the production method thereof.

Examples of the human iPS cell line include, specifically, 253G1 strain (iPS cell line prepared by expressing OCT4/SOX2/KLF4 in skin fibroblast of 36-year-old female), 201B7 strain (iPS cell line prepared by expressing OCT4/SOX2/KLF4/c-MYC in skin fibroblast of 36-year-old female), 1503-iPS (297A1) (iPS cell line prepared by expressing OCT4/SOX2/KLF4/c-MYC in skin fibroblast of 73-year-old female), 1392-iPS (297F1) (iPS cell line prepared by expressing OCT4/SOX2/KLF4/c-MYC in skin fibroblast of 56-year-old male), NHDF-iPS (297 L1) (iPS cell line prepared by expressing OCT4/SOX2/KLF4/c-MYC in skin fibroblast of newborn boy) and the like.

### IV. Cell Culture Method

Included herein are methods for differentiating a stem or progenitor cell or a starting population of stem and/or progenitor cells into neural cells. The methods of the disclosure may comprise or further comprise culturing the cells in a medium as defined herein and/or comprising or further comprising supplements and components described herein. These methods are discussed in more detail below. It is further specifically contemplated that the methods of the disclosure may exclude culturing the cells in medium or with supplements described herein.

The cell culture may be a 3-dimensional (3D cell culture). The 3D environment provides a balance between accumulation of paracrine factors and renewal of nutrients in the extracellular matrix. The derivation of neurons in 3D has also the advantages of i) using low volumes of media and consequently less amounts of novel neuroactive substances used for the assay, and ii) the option for multiplexing (96-well plate format), which is essential for automated screening activities.

To achieve the three dimensional property of the cell culture, cells can be grown or differentiated in matrices, scaffolds, or microwells. In principle, suitable matrices or scaffolds, which can be used in three dimensional cell cultures are known to the skilled artesian. Such matrices or scaffolds can therefore be any matrix or scaffold. For example, the matrix or scaffold can be an extracellular matrix comprising either natural molecules or synthetic polymers, a biological and synthetic hybrid, metals, ceramic and bioactive glass or carbon nanotubes.

Exemplary natural extracellular matrix molecules include collagen, basement membranes such as laminin or fibrin, alginates, chitosan, hyaluronic acid, silk fibroin, cellulose acetate, casein, chitin, fibrinogen, gelatine, elastin or poly-(hydroxyalkanoate). Synthetic extracellular matrix polymers include hyaluronic acid (HA) modified forms, poly-ethylen glycol (PEG) modified forms, self-assembling protein hydrogels, poly(lactic-co-glycolic acid) (PLGA), polycaprolactone (PCL), polyurethane or PGS. Biological and synthetic hybrids can for example include polycaprolactone-chitosan, PLLA-Hydroxyapatite, hydroxyapatite-bioglass-ceramic, poly-(hydroxylalkanoate)-bioglass, hydroxyapatite-collagen, PCL-gelatin or PCL-collagen. Exemplary metals include tantalam, magnesium and its alloys, titanium and its alloys or nitinol (nickel and titanium alloys). Examples of ceramics and bioactive glass matrices/scaffolds include titanium and tri calcium phosphate, hydroxyapatite and tricalcium phosphate, bioactive silicate glass (SiO₂—Na₂O—CaO—P₂O₅), hydroxyapatite and bioglass, calcium phosphate glass or phosphate glass. Carbon nanotubes can be constructed using graphite ranging from 0.4 to 2 nm. Carbon nanotubes can comprise CNT-polycaprolactone, CNT-ceramic matrix, 45S5 bioglass-CNT, CNT studded with gelatin hydrogel, CNT-TiO₂, CNT-laminin, CNT grafted with polyacrylic acid or CNT-TGF-beta

The matrix or scaffold can also be a hydrogel such as matrigel, fibrin gel or alginate gel. Optionally growth factors and other molecules can be added to the matrigel. The matrigel can also be mixed with a medium. E.g. the matrigel can be diluted with a medium as described herein or in the Examples. The matrigel can also be BD Matrigel™

The three-dimensional cell culture used in the present invention may exclude an organoid culture. Organoids are three-dimensional tissue structures, often generated from pluripotent stem cells (PSCs) but e.g. also from neuroepithelial stem cells, which self-organize and recapitulate complex aspects of their organ counterparts, ranging from physiological processes to regeneration and disease.

The methods may comprise contacting cells or spheroids with or the differentiation medium may comprise a Hedgehog signaling pathway activator. The “Hedgehog signaling pathway” or “SHH pathway” is well known in the art and has been described, for example, in Choudhry et al. (2014) “Sonic hedgehog signaling pathway: a complex network.” Ann Neurosci. 21(1):28-31. Hedgehog ligands, including, for example, Sonic hedgehog, Indian hedgehog, and/or Desert hedgehog, bind to the receptor, including, for example, Patched or the patched-smoothened receptor complex, which induces a downstream signaling cascade. Downstream target genes of SHH signaling include GLI1, GL12 and/or GL13. Accordingly, the term “activator of the Hedgehog signaling pathway” also refers to an activator of any one of the above recited molecules that form part of this signaling pathway.

The methods may comprise contacting cells or spheroids with or the differentiation medium may comprise an activator of SHH. Exemplary activators of the Sonic hedgehog (SHH) signaling include purmorphamine (PMA; 2-(1-Naphthoxy)-6-(4-morpholinoanilino)-9-cyclohexylpurine 9-Cyclohexyl-N-[4-(4-morpholinyl)phenyl]-2-(1-naphthalenyloxy), CAS No.: 483367-10-8), SHH, smoothened agonist (SAG; 3-chloro-N-[trans-4-(methylamino)cyclohexyl]-N-[[3-(4-pyridinyl)phenyl]me-thyl]-benzo[b]thiophene-2-carboxamide, CAS No.: 912545-86-9) and Hh-Ag 1.5 (3-chloro-4,7-difluoro-N-(4-(methylamino)cyclohexyl)-N-(3-(pyridin-4-yl) benzyl)benzo[b]thiophene-2-carboxamide; CAS No.: 612542-14-0). The SHH-pathway activator can therefore be purmorphamine. The SHH pathway activator can also be a recombinant or truncated form of SHH, which retains SHH pathway activating functions such as e.g. SHH C24II.

The methods may comprise contacting cells or spheroids with or the differentiation medium may comprise neurotrophins. The term “neurotrophins”, as used herein, relates to a family of proteins that regulate the survival, development, and function of neurons. Exemplary neurotrophins include Insulin-like growth factor 1 (IGF), Fibroblast growth factors (FGF), Transforming growth factor beta (TGF), Leukemia inhibitory factor (LIF), nerve growth factor (NGF), brain-derived neurotrophic factor (BDNF), neurotrophin-3 (NT-3), neurotrophin-4 (NT-4) as well as GDNF family of ligands and ciliary neurotrophic factor (CNTF). The GDNF family of ligands includes glial cell line-derived neurotrophic factor (GDNF), neurturin (NRTN), artemin (ARTN), and persephin (PSPN).

The methods may comprise contacting cells or spheroids with or the differentiation medium may comprise an antioxidant. An antioxidant is a molecule that inhibits the oxidation of other molecules. The terms “oxidation” and “antioxidant” are well known in the art and have been described, for example, in Nordberg J, Arner E S. (2001) “Reactive oxygen species, antioxidants, and the mammalian thioredoxin system.” Free Radic Biol Med. 31(11):1287-312. In short, oxidation is a chemical reaction involving the loss of electrons or an increase in oxidation state. Oxidation reactions can produce free radicals. In turn, these radicals can start chain reactions. When the chain reaction occurs in a cell, it can cause damage or death to the cell. Antioxidants terminate these chain reactions by removing free radical intermediates, and inhibit other oxidation reactions. Accordingly, an antioxidant refers to an inhibitor of a molecule involved in cellular oxidative processes.

Exemplary antioxidants include ascorbic acid, superoxide dismutase 1, superoxide dismutase 2, superoxide dismutase 3, glutathione, lipoic acid, epigallocatechin gallate, curcumine, melatonin, hydroxytyrosol, ubiquinone, catalase, vitamin E or uric acid. Thus, the antioxidant can be ascorbic acid.

The methods may comprise contacting cells or spheroids with or the differentiation medium may comprise an activator of activin/transforming growth factor-beta (TGF-beta) signaling pathway. The activin/TGF-beta signaling pathway is known in the art and include, for example, receptor ligands, including, for example, TGFB1, TGFB2, TGFB3, ACTIVIN A, ACTIVIN B, ACTIVIN AB, and/or NODAL, bind to a heterotetrameric receptor complex consisting of two type I receptor kinases, including, for example, TGFBR2, ACVR2A, and/or ACVR2B, and two type II receptor kinases, including, for example, TGFBR1, ACVR1 B, and/or ACVR1C. Exemplary activators of the activin/TGF-beta3 signaling pathway include TGFbeta1, TGFbeta2, TGFbeta3, activin A, activin B, activin AB or nodal. Thus, the activator of activin/TGF-beta signaling pathway can be TGFbeta3.

The methods may comprise contacting cells or spheroids with or the differentiation medium may comprise a cAMP analogue. Such cAMP analogs are compounds that have similar physical, chemical, biochemical, or pharmacological properties as the cyclic adenosine monophosphate (cAMP). Exemplary cAMP analogues include forskolin, 8-(4-chloro-phenylthio)-2′-O-methyladenosine-3′,5′-cyclic monophosphate (8CPT-2Me-cAMP), 8-Chloro-cAMP (8-Cl-cAMP), Bucladesine, Rp-adenosine .3., 5.,-cyclic monophosphorothioate sodium salt (Rp-cAMPS), Sp-8-hydroxyadenosine .3., 5.,-cyclic monophosphorothioate sodium salt (Sp-80H-cAMPS) and Rp8-hydroxyadenosine .3., 5.,-cyclic monophosphorothioate sodium salt (Rp-80H-cAMPS) or dbcAMP. Thus, the cAMP analogue can be dbcAMP.

The methods may comprise contacting cells or spheroids with or the differentiation medium may comprise a N2B27 medium (into which the different compounds are diluted). This means that the medium comprises a N2 supplement and a B27 supplement. Both supplements are well known to the person skilled in the art and freely available. The B27 supplement can be a B27 supplement without vitamin A.

The differentiation medium may comprise a Neurobasal medium and/or a DMEM-F12 medium. Both media can for example be obtained from Gibco. The N2B27 medium can for example comprise equal amounts of Neurobasal medium and DMEM/F12 medium.

The cell culture medium may be one that is known and used in the art for culturing stem cells, such as STEMFLEX cell culture medium. The medium may comprise a medium to culture neurons, such as NEUROBASAL medium, NEUROBASAL-A medium, or Neural Progenitor Basal medium. The medium may comprise NS-A medium, BME medium, BGJb medium, CMRL 1066 medium, Glasgow MEM medium, Improved MEM Zinc Option medium, IMDM medium, Medium 199 medium, Eagle MEM medium, αMEM medium, DMEM medium, DMEM/F12 medium, ham medium, RPMI 1640 medium, Fischer's medium, and mixed medium thereof and the like. The cell culture mediums are generally available for purchase from Invitrogen, SIGMA, Wako Pure Chemical Industries, Ltd., Sumitomo Dainippon Pharma Co., Ltd. and the like.

The medium to be used in this differentiation method may be a serum-containing medium or a serum-free medium (such as KNOCKOUT medium). As used herein, the serum-free medium means a medium free of a non-adjusted or unpurified serum, and a medium containing purified blood-derived components and animal tissue-derived components (e.g., growth factor) corresponds to a serum-free medium. When the medium to be used in this differentiation method is a serum-containing medium, and a serum of a mammal such as fetal bovine serum and the like can be used as the serum. The concentration of the serum in the medium is generally 0.01-20 wt % or 0.1-10 wt %.

The medium to be used in this differentiation method may also contain a serum replacement. Examples of the serum replacement include albumin (e.g., lipid-rich albumin), transferrin, fatty acid, collagen precursor, trace element (e.g., zinc, selenium), B-27 supplement, N2 supplement, Replacement KnockOut serum replacement, 2-mercaptoethanol, 3′thiolglycerol, and equivalents thereof. The concentration of these in the media is the same as the concentration of the aforementioned serum in the medium.

The medium to be used in methods of the disclosure may also contain lipid, amino acid (e.g., non-essential amino acid), vitamin, growth factor, cytokine, antioxidant, 2-mercaptoethanol, pyruvic acid, buffering agent, inorganic salt, antibiotic (e.g., penicillin and streptomycin) or antibacterial agent (e.g., amphotericin B) and the like. The concentration of these in the media is the same as the concentration of the aforementioned serum in the medium.

Other culture conditions such as culture temperature, C02 concentration and the like can be appropriately determined. While the culture temperature is not particularly limited, it is, for example, about 30-40° C., preferably about 37° C. The C02 concentration is, for example, about 1-10%, preferably about 5%.

Methods may include the evaluation of cells, for example, an evaluation method of expression of protein by utilizing an antigen-antibody reaction, an evaluation method of gene expression by utilizing quantitative RT-PCR, and the like. Methods may also include evaluating the cells for expression of a cell marker, such as Pax6, Otx2, FoxA2, Lmx1a, and Msx1.

Cell culture conditions may be provided for the culture of neural or progenitor cells as provided herein. Starting cells of a selected population may comprise at least or about 10⁴, 10⁵, 10⁶, 10⁷, 10⁸, 10⁹, 10¹⁰, 10¹¹, 10¹², 10¹³ cells or any range derivable therein. The starting cell population may have a seeding density of at least or about 10, 10¹, 10², 10³, 10⁴, 10⁵, 10⁶, 10⁷, 10⁸ cells/ml, or any range derivable therein.

A culture vessel used for culturing the cells of the disclosure, or progeny cells thereof, can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, and roller bottle, as long as it is capable of culturing the cells therein. The cells may be cultured in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. The culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, laminin 521, fibronectin, and mixtures thereof for example Matrigel™, and lysed cell membrane preparations.

The culture methods may comprise culturing human stem cells, such as HS420 in Stemflex medium (ThermoFisher) on laminin 521-coated tissue culture flasks (ThermoFisher). At “Day 0” of differentiation, HS420 cells may be plated at 70% of confluency and passaged using Accutase™ (ThermoFisher). Cells can be suspended in X-VIVO medium (LONZA, ref BE04-380Q) supplemented with 1% of penicillin/streptomycin, ROCK inhibitor (Y27632, Abcam) at 10 M, and neural inducers (ALK inhibitors). 2200 human pluripotent stem cells per microwell in supplemented X-VIVO medium can be deposited in microwells plates. For example, on a substrate material consisting in molded microwells on an insert in air-liquid interface (720 microwells per well). Supplemented X-VIVO medium can be added under the insert. In order to correctly distribute the cells in each microwell, the substrate material can be gently agitated and put on a stable support for 15 minutes to let the cells sediment at the bottom of the wells. After these 15 minutes, the medium from the cell suspension can be removed by aspiration with a 0.5×16 mm needle and spheroids can be just covered by a thin film of residual medium before incubation of the plate at 37° C. for 24 hours.

On “Day 1” of differentiation, 24 hours after spheroid formation, culture medium under the insert can be replaced with fresh medium made by a combination of half X-VIVO medium (Lonza) and half Neurobasal medium (ThermoFisher) without ROCK inhibitor supplemented with one or more of 1% of penicillin/streptomycin, 1% Non-essential amino-acids, 1% B-27 supplement (ThermoFisher), L-Glutamin, 100 ng/ml of SHH, 2 μM of Purmorphamin and 100 ng/ml FGF-8 and neural inducers (ALK inhibitors).

On “Day 3” of differentiation, half of the medium may be changed to replace progressively the X-VIVO medium by the Neurobasal medium. The Neurobasal medium may comprise 1% of penicillin/streptomycin, 1% Non-essential amino-acids, 1% B-27 supplement (ThermoFisher), L-Glutamin, 100 ng/ml of SHH, 2 μM of Purmorphamin, 100 ng/ml FGF-8, 3 μM of CHIR99021 and neural inducers (ALK inhibitors).

On “Day 8” of differentiation, half of the medium may be replaced with Neurobasal medium supplemented with one or more of 1% of penicillin/streptomycin, 1% Non-essential amino-acids, 1% B-27 supplement, 3 μM of CHIR99021, 0.5 mM of cAMP, 20 ng/mL of GDNF, 20 ng/mL of BDNF, 5 ng/mL of FGF20, 1 ng/mL of TGF-B3 and 1 μM of Compound E.

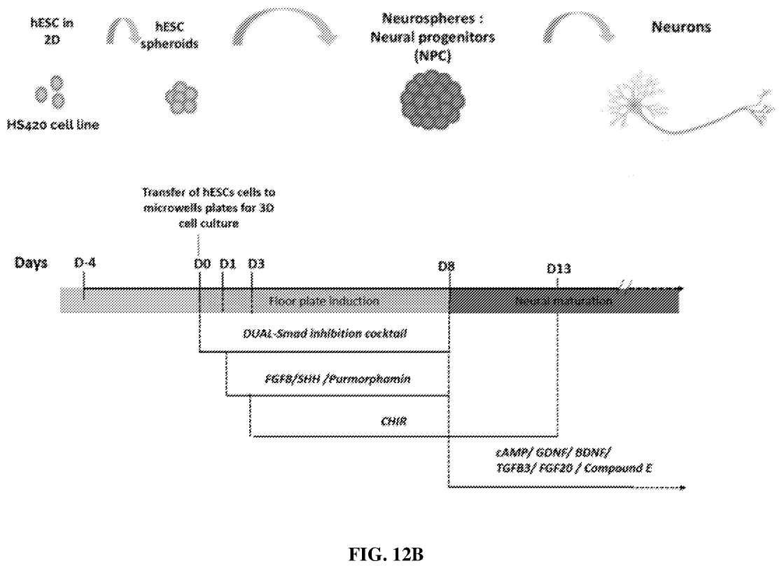

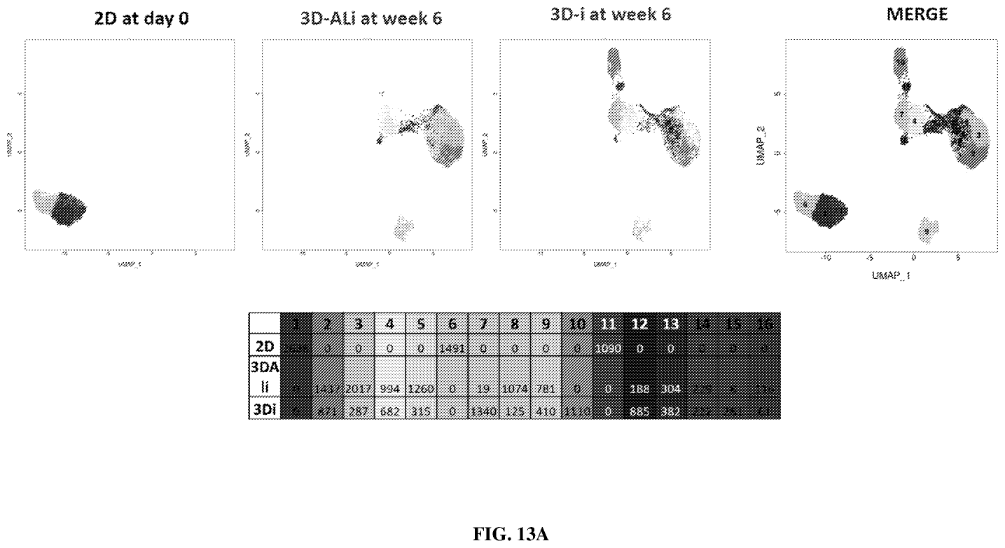

On “Day 13” of differentiation, half of the medium may be changed. CHIR99021 may be removed. Medium may be replaced by Neurobasal medium comprising one or more of with 1% of penicillin/streptomycin, 1% Non-essential amino-acids, 1% B-27 supplement, 0.5 mM of cAMP, 20 ng/mL of GDNF, 20 ng/mL of BDNF, 5 ng/mL of FGF20, 1 ng/mL of TGF-B3 and 1 μM of Compound E. FIG. 12 exemplifies 3-dimensional culture methods.

### V. Cell Culture Substrates

The methods include culturing cells in microwells. The microwells may be a part of a substrate. The substrate may comprise a hydrophilic and porous material layer with a plurality of lasting well-shaped indents on its outer surface supported by a semipermeable membrane on its inner surface, wherein said well-shaped indents have an aperture from 1 pm² to 1 mm² and a bottom surface from about 1 pm² to 1 mm².

The expression “semi-permeable membrane” refers to a membrane that allows the passage of solvent as well as a fraction of the solutes. It allows the preferential passage of certain substances present in a solution compared to others, depending on the size of the pores. It encompasses hydrophilic membranes, filter membranes and porous membranes.

The hydrophilic and porous material may be a hydrophilic gel allowing the diffusion of aqueous cell culture medium and not attaching to cells. Typically, a hydrophilic gel according may be made from a polymer selected from agarose, Polyvinyl(alcohol) (PVA), agar-agar, alginates, hyaluronic acid, pectin or starch or any equivalent synthetic gel.

A hydrophilic gel according may be made from a polymer from cell compatible naturally (or modified) derived materials such as from carbohydrates (e.g. agarose, agar-agar, alginate, starch, pectin, chitosan, dextran, polysaccharides), proteins or ECM components (e.g. laminins, collagens, hyaluronan, fibrin), peptides (e.g. gelatin), and mixtures thereof; or a gel derived from natural ECM, preferably Matrigel™ (a reconstituted basement membrane preparation that is extracted from mouse sarcoma, a tumor rich in extracellular matrix proteins (this material, once isolated, contains approximately 60% laminin, 30% collagen IV, and 8% entactin. Entactin is a bridging molecule that interacts with laminin and collagen IV and contributes to the structural organization of these extracellular matrix molecules. Coming Matrigel™ matrix also contains heparan sulfate proteoglycan (perlecan), transforming growth factor (TGF-beta), epidermal growth factor (EGF), insulin-like growth factor (IGF-1), fibroblast growth factor (bFGF), tissue plasminogen activator, and other growth factors which occur naturally in the EHS tumor. There are also residual matrix metalloproteinases derived from the tumor cells), Myogel (a human-based extracellular matrix myogel is extracted from human benign tumor tissue “leiomyoma” as described in Abberton et al., 2008, Cells Tissues Organs, 188(4):347-58. doi: 10.1159/000121575. Epub 2008 Mar. 20. PMID: 18354248, ‘Salo, et al., 2015, BMC Cancer, 15, 981 https: doi.or 10.1186/sl 2885-015-1944-z, or Cartigel (an extracellular matrix extract of cartilage) or synthetic derived materials selected from polyethylene glycol), polyaliphatic polyurethanes, polyether polyurethanes, polyethylene copolymers, polyamides, polyvinyl alcohols, poly(ethylene oxide), polypropylene oxide, polyethylene glycol, polypropylene glycol, polytetramethylene oxide, polyvinyl pyrrolidone, polyacrylamide, poly(hydroxy ethyl acrylate), poly(hydroxyethyl methacrylate), and mixtures thereof.

The hydrophilic gel can be prepared by many methods known in the art and involve covalent crosslinking or non-covalent assembly, or a combination of both. Covalent crosslinking can further be carried out as part of the synthesis process, for instance by using bifunctional monomers during polymerization, or alternatively by crosslinking of pre-formed macromolecules of hydrophilic polymers, wherein the polymers are linear or branched, using natural or synthetic macromolecules in the process.

A malleable layer of a hydrophilic and porous material may be a layer of a hydrophilic gel formed at a temperature above the gelling temperature of the gel (e.g. from 20° C. to 100° C. above the said gelling temperature). Typically, a malleable layer of agarose is formed by depositing agarose at a temperature between 75° C. and 100° C. on the semipermeable membrane to form a composite matrix. A malleable layer of a hydrophilic gel may be from about 5 mm to 10 cm large and from about 2 mm to 10 mm thick when above the gelling temperature. Hydrophilic gels may prevent adhesion of cells and of spheroids, once formed, thereby avoiding the use of an anti-adherent solution prior to centrifugation during cell culture. A malleable layer of a hydrophilic and porous material may have a porosity which allows the passage of oxygen and cell nutrients. The porosity may range from about 10 nm to about 500 pm. A malleable layer of a hydrophilic and porous material, such as agarose, may have a porosity range from about 100 nm to about 500 mm, which may be mostly occupied by water molecules, allowing the passage of nutrients, small molecules, chemical compounds and even proteins such as albumin.

The substrate may have between 4 to 1,536 well-shaped indents or may have, have at least, or have at most 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, 1000, 1000, 1100, 1200, 1300, 1400, 1500, 1600, 1700, 1800, 1900, or 2000 (or any derivable range therein) well-shaped indents.

The formation of spheroids can be achieved under over time by gravity from about 2h to 5h or by subjecting the cell culture vessel containing the cell seeded floating substrate material to centrifugation to induce cell aggregation. The cell culture vessel may be centrifuged at 100×g for a period from 5 to 10 minutes.

### VI. Neural Cells

Methods include the differentiation of cells into neural cells. The methods may be used to generate one or more types of neural cells including motor neurons, sensory neurons, and interneurons. A typical neuron consists of a cell body (referred to as a soma), dendrites, and an axon. The methods may be used to generate cholinergic neurons, GABAergic neurons, motor neurons, astrocytes, oligodendrocytes, glutamatergic neurons, dopaminergic neurons, and/or serotonergic neurons. The methods may be used to generate dopaminergic neurons. The methods may include differentiating neural cells into neurons, astrocytes, oligodendrocytes, dopamine neurons, or motor neurons, pyramidal neurons, motor neurons, spinal ventral horn motor neurons, neurons of the ventral mesencephalon, interneurons, glial cells, radial glial cells, retinal pigment epithelium, oligodendrocytes, dopamine neurons, GABA neurons, glutamate neurons, catecholinergic neurons, serotoninergic neurons, and cholinergic neurons.

Methods may also include the evaluation of progenitor and/or neural cells. For example, the production or differentiation of neural cells in a cell population may be determined through the presence of certain cell markers. Those markers may vary depending on the species or organism that is used for the starting population. Examples of neural cell markers in organisms such as humans include transcription factors or structural proteins. Examples of transcription factors include MYT1L, BRN2, SOX1, PAX6, NKX6.1, OLIG2, NGN2, LHX3, ISL1/2, and HB9. Other neural markers include tubulin (e.g., Tubb2a and Tubb2b), Map2, Synapsin (e.g., Syn1 and Syn2), synaptophysin, synaptotagmins (e.g., Syt1, Syt4, Syt13, Syt 16), NeuroD, cholineacetyltransferase (ChAT) (e.g., vesicular ChAT), neurofilament, neuromelanin, Tuj1, Thy1, Chat, GluR (kainite 1), Neurod 1, and the like. Expression of receptors for excitatory and inhibitory neurotransmitters can also be used to assess the number and quality of neural cells generated. In addition, gross cell morphology may be used to identify neural cells in a population of non-neural cells. The neural cells of the disclosure may also exclude one or more of the markers listed herein such as MYT1L, BRN2, SOX1, PAX6, NKX6.1, OLIG2, NGN2, LHX3, ISL1/2, and HB9, Tubb2a, Tubb2b, Map2, Synapsin, Syn1, Syn2, synaptophysin, synaptotagmins, Syt1, Syt4, Syt13, Syt 16, NeuroD, cholineacetyltransferase (ChAT), vesicular ChAT, neurofilament, neuromelanin, Tuj1, Thy1, Chat, GluR (kainite 1), and Neurod 1.

The presence of neural cells may also be assessed functionally. For example, the cells may be assessed according to electrophysiological characteristics. These assessments may be made using patch-clamp recordings. Other functional characteristics include ability to fire action potentials, produce an outward current in response to glycine, GABA or kainite, and produce an inward current in response to glutamate.

Neural cells may be assessed and thus identified by the presence of one or more, including 2, 3, 4, 5, or more, of any of the foregoing characteristics and/or markers.

The neural cells or cell population may also be assessed for expression of markers characteristic of the non-neural starting cell population. Reprogramming, in some instances, may be evaluated by the increased expression of neural markers and decreased expression of markers of the non-neural starting cells.

The neural cell may be further defined as a dopaminergic (DA) neuron. DA neurons can be confirmed by evaluating the expression variation of proteins and genes that are specifically expressed by the dopaminergic neuron (in the present specification, the above-mentioned proteins and genes are sometimes referred to as a dopaminergic neuron marker). The above-mentioned evaluation of expression variation of dopaminergic neuron cell marker can be performed by, for example, an evaluation method of expression of protein by utilizing an antigen-antibody reaction, an evaluation method of gene expression by utilizing quantitative RT-PCR and the like. Examples of the above-mentioned dopaminergic neuron cell marker, which is present in the midbrain, include tyrosine hydroxylase (TH), OTX2, FOXA2, LMX1A, LMX1B, PITX3, EN1 and NURR1 gene/protein.

In addition, whether the dopaminergic neuron obtained by the production method of the present invention has functions equivalent to those of dopaminergic neuron in vivo can be confirmed by evaluating dopamine release, and responsiveness to oxidative stress and drug stimulation.

The cells obtained during the processes of the production method of the present invention and the dopaminergic neuron of the present invention can be cryopreserved and thawed. Freezing and thawing methods of cells are known in the pertinent field, and are not particularly limited as long as they do not influence differentiation potency, viability, dopamine production capability and the like of the cells. For example, the dopaminergic neuron of the present invention can be preserved at −80° C. by washing cells with PBS, detaching same from a culture dish with a cell-dispersion solution (e.g., Accutase (registered trade mark) Innovative Cell Technologies), removing the cell-dispersion solution, and suspending the cells in a cryopreservation solution (e.g., cell banker 2 (LSI Medience Corporation)). Examples of the thawing method include a method comprising thawing in a thermostatic tank at 37° C., washing a cryopreservation solution by centrifugation, and suspending in a medium for use, and the like. When the cells obtained during the processes of the production method of the present invention are frozen and thawed, Nurr1 positive dopaminergic neuron can also be induced from the cells after thawing.

### VII. Methods of Use of the Neural Cells

Methods of the disclosure relate to the production of a neural progenitor cells that may be, used for treatment of subjects. For example, the cells produced by methods of the disclosure may be used to treat neurodegenerative diseases. Non-limiting examples of neurodegenerative diseases include Alzheimer disease; epilepsy; Huntington's Disease; Parkinson's Disease; stroke; spinal cord injury; traumatic brain injury; Lewy body dementia; Pick's disease; Niewmann-Pick disease; amyloid angiopathy; cerebral amyloid angiopathy; systemic amyloidosis; hereditary cerebral hemorrhage with amyloidosis of the Dutch type; inclusion body myositis; mild cognitive impairment; Down's syndrome; and neuromuscular disorders including amyotrophic lateral sclerosis (ALS), multiple sclerosis, and muscular dystrophies including Duchenne dystrophy, Becker muscular dystrophy, Facioscapulohumeral (Landouzy-Dejerine) muscular dystrophy, and limb-girdle muscular dystrophy (LGMD). Also included is neurodegenerative disease due to stroke, head trauma, spinal injury, or other injuries to the brain, peripheral nervous, central nervous, or neuromuscular system. The methods set forth herein may pertain to methods of preventing a disease or health-related condition in a subject.

The current disclosure provides a medicament containing a neuron produced by the methods of the disclosure. As used herein, the neuron is not particularly limited as long as it is a cell obtained by the above-mentioned production method of the disclosure.

In this medicament, a neuron may be used as is, or as a cell aggregate obtained by concentration by passing through a filter and the like, such as pellet and the like. Furthermore, the medicament can also be added with a protector such as DMSO (dimethyl sulfoxide) and the like and cryopreserved. For safer utilization of the medicament, the medicament may be subjected to a treatment under such conditions as to retain the function of the neuron and denature pathogenic protein, for example, heat treatment, radiation treatment and the like. Moreover, to prevent growth of the neuron in an amount more than necessary, the medicament may be subjected to, in combination with the above-mentioned treatments, suppression of growth by a mitomycin C pre-treatment and the like, and a treatment by a method including introducing a gene of a metabolic enzyme naturally absent in mammals into the neurons, administering an agent in an inactivated form as necessary to allow for the agent to be converted to a toxicant only in the neurons, into which the gene of a metabolic enzyme naturally absent in mammals has been introduced, thus leading the cells to eradication (suicide gene therapy) and the like.

Since the medicament of the current disclosure is safe and has low toxicity, it can be administered to a mammal (e.g., human, mouse, rat, guinea pig, swine, monkey).

A neural cell may be prepared using a patient's own cell or a cell of a donor having a histocompatibility type in a tolerable range is used for the medicament of the current disclosure. When sufficient cells cannot be obtained due to age, constitution and the like, the cells embedded with a polyethylene glycol or silicon capsule, a porous container and the like can also be transplanted to avoid rejection. The dose (amount to be transplanted) and administration frequency (number of times to be transplanted) of the medicament of the present disclosure can be appropriately determined according to the age, body weight, symptom and the like of the patients who receive administration.

A medicament containing the neuron of the disclosure can efficiently engraft in the body of patients by administration (transplantation) thereof, which in turn enables efficient production (release) of dopamine in the body of patients. Therefore, the medicament of the disclosure is useful for the treatment of diseases caused by decreased production (release) of dopamine, for example, neurodegenerative diseases such as Parkinson's disease, Huntington chorea, Alzheimer's disease, epilepsy and schizophrenia and the like.

The neural cells of the disclosure may be used in a method for screening for a drug compound, such as a compound for the treatment of neurodegenerative diseases. For example, whether the test compound is useful as a medicament can be evaluated by contacting the test compound alone or in combination with other medicament with the neuron of the disclosure, and measuring morphological or functional change of the neuron. Examples of the method for measuring the functional change include measuring the amount of dopamine produced or released from the neuron. The dopaminergic neuron may be a cell showing the same phenotype as the disease to be the treatment target, and particularly preferred is a dopaminergic neuron produced by inducing differentiation of a stem cell produced from a somatic cell derived from the disease.

Examples of the test compound include peptide, protein, antibody, nonpeptidic compound, synthetic compound, fermentation product, cell extract, plant extract, animal tissue extract, plasma and the like. As used herein, the test compound may form a salt. As the salt, a salt with a physiologically acceptable acid (e.g., inorganic acid, organic acid), a base (e.g., alkali metal salt, alkaline earth metal salt, aluminum salt) and the like is used, and examples of such salt include a salt with an inorganic acid (e.g., hydrochloric acid, phosphoric acid, hydrobromic acid, sulfuric acid), a salt with an organic acid (e.g., acetic acid, formic acid, propionic acid, fumaric acid, maleic acid, succinic acid, tartaric acid, citric acid, malic acid, oxalic acid, benzoic acid, methanesulfonic acid, benzenesulfonic acid), sodium salt, potassium salt, calcium salt, magnesium salt, barium salt, and aluminum salt can be used.

The medicament obtained using the above-mentioned screening can be formulated using a physiologically acceptable additive and according to a known method.

### VIII. Additional Agents

It is contemplated that methods of the disclosure include the administration of additional agents. The additional agent may comprise one or more BMP inhibitors such as Noggin, chordin, dorsomorphin, LDN-193189 (4-(6-(4-(piperazin-1-yl)phenyl)pyrazolo[1,5-a]pyrimidin-3-yl)quinoline hydrochloride), dorsomorphin(6-[4-(2-piperidin-1-ylethoxy)phenyl]-3-pyridin-4-ylpyrazolo[1,5-a]pyrimidine) and the like; TGFβ family inhibitors such as SB431542 (4-[4-(1,3-benzodioxol-5-yl)-5-(2-pyridinyl)-1H-imidazol-2-yl]-benzamide), A-83-01 (3-(6-methylpyridin-2-yl)-1-phenylthiocarbamoyl-4-quinolin-4-ylpyrazole) and the like; GSK3B inhibitors such as CHTR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), and BIO (6-bromo-indirubin-3′-oxime); Smoothened agonists such as purmorphamine (N-(4-morpholinophenyl)-2-(1-naphthyloxy)-9-cyclohexyl-9H-purin-6-amine), and SAG (N-methyl-N′-(3-pyridinylbenzyl)-N′-(3-chlorobenzo[b]thiophene-2-carbonyl)-1,4-diaminocyclohexane); and specification factors such as Sonic hedgehog (SHH) and fibroblast growth factor-8 (FGF8). The methods may exclude contact of the cells with one or more of the additional agents described herein. The methods may exclude contact of the cells with one or more BMP inhibitors such as chordin, dorsomorphin, Noggin, LDN-193189 (4-(6-(4-(piperazin-1-yl)phenyl)pyrazolo[1,5-a]pyrimidin-3-yl)quinoline hydrochloride), dorsomorphin(6-[4-(2-piperidin-1-ylethoxy)phenyl]-3-pyridin-4-ylpyrazolo[1,5-a]pyrimidine) and the like; TGFβ family inhibitors such as SB431542 (4-[4-(1,3-benzodioxol-5-yl)-5-(2-pyridinyl)-1H-imidazol-2-yl]-benzamide); GSK30 inhibitors such as CHIR99021 (6-[[2-[[4-(2,4-dichlorophenyl)-5-(5-methyl-1H-imidazol-2-yl)-2-pyrimidinyl]amino]ethyl]amino]-3-pyridinecarbonitrile), and BIO (6-bromo-indirubin-3′-oxime); Smoothened agonists such as purmorphamine (N-(4-morpholinophenyl)-2-(1-naphthyloxy)-9-cyclohexyl-9H-purin-6-amine), and SAG (N-methyl-N′-(3-pyridinylbenzyl)-N′-(3-chlorobenzo[b]thiophene-2-carbonyl)-1,4-diaminocyclohexane); and growth factors such as Sonic hedgehog (SHH) and fibroblast growth factor-8 (FGF8).

The additional agent may include one or more of an activator of the phosphatidylinositol 3-kinase signaling pathway and an activator of the MAPK signaling pathway. The additional agent may exclude one or more of an activator of the phosphatidylinositol 3-kinase signaling pathway and an activator of the MAPK signaling pathway. The additional agent can comprise one or more of Midkine, Pleiotrophin, insulin-like growth factor-1, an inhibitor of the TGF-P superfamily signaling pathway, A83-01, SB431542, dorsomorphin, an inhibitor of the Wnt signaling pathway, PNU-74654, Dickkopf, an activator of the Notch signaling pathway such as Delta-1, Delta-2, Delta-3, Delta-4, Jagged-1, Jagged-2, an activator of the protein kinase signaling pathway such as Forskolin, or dibutyryl cAMP, an activator of tyrosine kinase anaplastic lymphoma kinase (ALK), and activator of insulin-like growth factor (IGF) receptor, and inhibitor of SMAD2, SMAD3, SMAD4, SMAD1, SMAD5, SMAD8, an inhibitor of Wnt or LRP binding to Frizzled, or an inhibitor of β-catenin stabilization. The methods may exclude contact of cell described herein with one or more of an activator of the phosphatidylinositol 3-kinase signaling pathway and an activator of the MAPK signaling pathway. The additional agent may excludes one or more of an activator of the phosphatidylinositol 3-kinase signaling pathway and an activator of the MAPK signaling pathway. The additional agent can comprise one or more of Midkine, Pleiotrophin, insulin-like growth factor-1, an inhibitor of the TGF-P superfamily signaling pathway, SB431542, an inhibitor of the Wnt signaling pathway, PNU-74654, Dickkopf, an activator of the Notch signaling pathway such as Delta-1, Delta-2, Delta-3, Delta-4, Jagged-1, Jagged-2, an activator of the protein kinase signaling pathway such as Forskolin, or dibutyryl cAMP, an activator of tyrosine kinase anaplastic lymphoma kinase (ALK), and activator of insulin-like growth factor (IGF) receptor, and inhibitor of SMAD2, SMAD3, SMAD4, SMAD1, SMAD5, SMAD8, an inhibitor of Wnt or LRP binding to Frizzled, or an inhibitor of 0-catenin stabilization.

### IX. Examples

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Three Dimensional Cell Culture for In Vitro Differentiation of Stem or Progenitor Cells into Neural Cells

### A. Material and Methods

### 1. Chemicals, Proteins and Antibodies

LDN193189 was provided by Axonmedchem (ref 1509), stored at −20° C. at the 5 concentration of 5 mM in DMSO. SB431542 was provided by Abcam (ref abi20163), stored at −20° C. at the concentration of 50 mM in DMSO. FGF-8 (fibroblast growth factor 8, ref GFH176-5) was provided by Peprotech, stored at −20° C. at the concentration of 100 μg/mL in pure water. SHH (Sonic Hedgehog protein, ref GFH168-5) was provided by Cell Guidance, stored at −20° C. at the concentration of 100 μg/mL in pure water. Purmorphamin (ref SML0868) was provided by Calbiochem, stored at −20° C. at the concentration of 10 mM in pure DMSO. CHIR99021 (ref ct99021) was provided by Axon Medchem, stored at −20° C. at the concentration of 7.5 mM in DMSO. cAMP (Cyclic adenosine monophosphate, ref D0627) was provided by Sigma, stored at −20° C. at the concentration of 0.5 M in DMSO. BDNF (Brain-derived neurotrophic factor, ref GFH1-2) was provided by Cell Guidance, stored at −20° C. at the concentration of 100 μg/mL in pure water. GDNF (Glial-derived neurotrophic factor, ref GFH2-2) was provided by Cell Guidance, stored at −20° C. at the concentration of 100 μg/mL in pure water. TGFB3 (transforming growth factor beta 3, ref GFH109-2, was provided by Cell Guidance, stored at −20° C. at the concentration of 100 μg/mL in pure water. FGF20 (Fibroblast growth factor 20, ref 100-41) was provided by Peprotech, stored at −20° C. at the concentration of 100 μg/mL in pure water. Compound E, a 7-secretase inhibitor (ref CAS 209986-17-4) was provided by Calbiochem, stored at −20° C. at the concentration of 5 mM in pure DMSO. For immunostainings, the following antibodies were used: mouse anti-111-tubulin (Sigma), rabbit polyclonal anti-TH (Merck), goat-anti-mouse IgG—Alexa 555 (Life technologies), goat anti-rabbit IgG—Alexa 488 (Life technologies).

### 2. Embryonic Stem Cell Culture and Differentiation

The human embryonic stem (hESC) cell line HS420 (Gift from Dr Outi Hovatta, Karolinska institute, Sweden) was cultured in Stemflex medium (Thermofisher) on laminin 521-coated tissue culture flasks (Thermofisher) according manufacturer's instructions. HS420 cells at 70% of confluency were passaged in 3D cell culture plates in X-VIVO medium (LONZA, ref BE04-380Q) supplemented with 1% of penicillin/streptomycin, ROCK inhibitor (Y27632, abeam) at 10 M, LDN193189 at 0.5 M and SB431542 at 10 M.

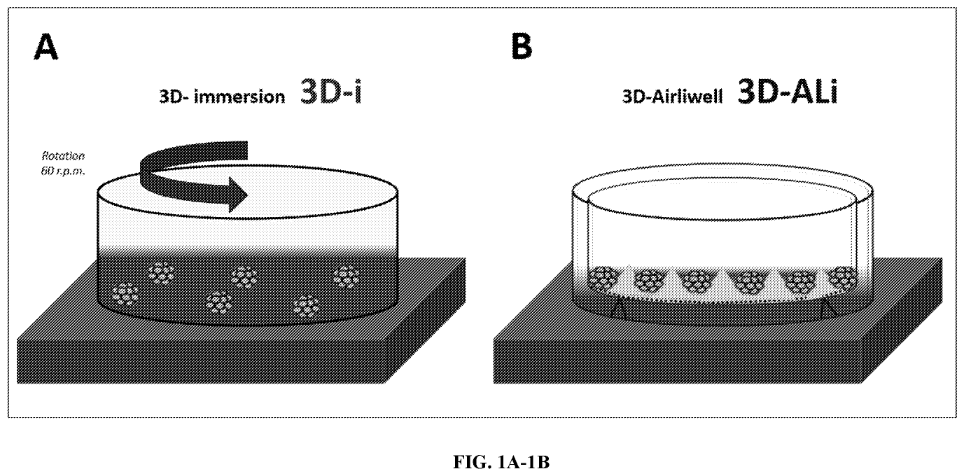

The dopaminergic neurospheres were manufactured by using the following two different 3D cell culture techniques. The first one was the standard method “3D-Immersion”, the spheroids are first forced aggregated in plastic microwells, then removed and transferred in standard culture plates maintained under agitation (FIG. 1A). In the second technique used, called “3D-AirLiwell” method, the spheroids are forced to aggregation in non-adhesive microwells molded in medium-permeable agarose. In contrast to 3D-Immersion, they are individually maintained into the microwells without any transfer, immersion and agitation. A hemi-permeable membrane at the bottom of the molded microwells allows their long-term stability and also establish air/liquid interface conditions that favor gas exchanges with air (FIG. 1B).

a. Manufacturing of Dopaminergic Neurospheres by Forced Aggregation Followed by Immersion (3Di)—

HS420 cells were deposited in supplemented X-VIVO medium in Aggrewell-800™ (Stemcell technologies, 6 well plate, 2 ml per well) at the ratio of 2′200 cells per microwell (the microwell plate used here contains 1′800 microwells per well). In order to correctly distribute the cells in each microwell, the plate is gently shacked and put on a stable support. After 15 min, the plate is cultured at 37° C. for 24 h to generate spheroids. 24 hours after spheroids formation, the spheres are collected and then transferred in a standard 6 well-plate in a combination of half X-VIVO and half Neurobasal medium without ROCK inhibitor supplemented with 1% of penicillin/streptomycin, 1% Non-essential amino-acids, 1% B-27 supplement (Thermofisher), L-Glutamin, 0.5 M of SB431542, 10 M LDN193189 (dual-SMAD inhibition cocktail), 100 ng/ml of SHH, 2 μM of Purmorphamin and 100 ng/ml FGF-8. Spheroids are then cultured under constant agitation in 3 ml of medium per well, and cultured at 37° C. and 5% C02 under constant agitation (60 rpm, orbital shaker). At day 3, 3 μM of CHIR99021 was added. The neurobasal medium replaced progressively the X-VIVO medium. At day 8, medium is replaced by Neurobasal medium supplemented with 1% of penicillin/streptomycin, 1% Non-essential amino-acids, 1% B-27 supplement, 3 μM of CHIR99021, 0.5 mM of cAMP, 20 ng/mL of GDNF, 20 ng/mL of BDNF, 5 ng/mL of FGF20, 1 ng/mL of TGF-B3 and 1 μM of Compound E. At day 13 CHIR99021 is removed. NB: for each medium exchange, the half of medium was refreshed, using between 1.5 and 2 ml per well.

b. Manufacturing of Dopaminergic Neurospheres by Using 3D-AirLiwell Technology(3D-ALi)—

For neural differentiation, 2200 human pluripotent stem cells per microwell in supplemented X-VIVO medium were deposited on a substrate material consisting in molded microwells on an insert in air-liquid interface (720 microwells per well). In order to correctly distribute the cells in each microwell, the substrate material is gently shacked and put on a stable support for 15 min. Supplemented X-VIVO medium was added under the insert. Medium from the cell suspension added on the microwells, was then removed by aspiration with a 0.5×16 mm needle and spheroids are just covered by a thin film (10) of residual medium before incubation of the plate at 37° C. for 24 hours.

24 hours after spheroids formation, culture medium under the insert is replaced with fresh medium made by a combination of half X-VIVO and half Neurobasal medium without ROCK inhibitor supplemented with 1% of penicillin/streptomycin, 1% Non-essential amino-acids, 1% B-27 supplement (Thermofisher), L-Glutamin, 0.5 μM of SB431542, 10 μM LDN193189 (dual-SMAD inhibition cocktail), 100 ng/ml of SHH, 2 μM of Purmorphamin and 100 ng/ml FGF-8. Spheroids are then cultured in their individualized microwells in a conventional incubator at 37° C. and 5% C02. At day 3, 3 μM of CHIR99021 was added. The neurobasal medium replaced progressively the X-VIVO medium. At day 8, medium is replaced by Neurobasal medium supplemented with 1% of penicillin/streptomycin, 1% Non-essential amino-acids, 1% B-27 supplement, 3 μM of CHIR99021, 0.5 mM of cAMP, 20 ng/mL of GDNF, 20 ng/mL of GDNF, 5 ng/mL of FGF20, 1 ng/mL of TGF-B3 and 1 μM of Compound E. At day 13 CHIR99021 is removed. NB: for each medium exchange, the half of medium was refreshed, using 0.5 ml per well.

### 3. Quantitative RT PCR and RNA Sequencing

For qRT-PCR, RNA was extracted from dopaminergic neurospheres cultivated in immersion (3D-i) and in air-liquid culture interface (3D-ALi) derived from 3 different cell batches of HS420 cells. The time points for RNA extraction were days: 3, 8, 15, 22, 29, 43, 57. Total RNA isolation was performed using the Qiagen RNeasy kit according to the manufacturer's instructions. RNA concentration was determined by a spectrometer (Thermo Scientific™ NanoDrop 2000) and RNA quality was verified by the 2100 Bioanalyzer (Agilent). Quantitative PCR was performed with Tecan Freedom Evo by using SYBR green fluorescence. cDNA was produced from RNA by using the prime script RT reagent kit (Takara). All primers were diluted at a concentration of 0.86 M, and a master mix was used (Power SYBR Green Master Mix, Thermo Fisher Scientific). To perform the RNA-sequencing, the same RNA used in qPCR, were used to analyze the gene expression at week 6 (=day 43) of differentiation.

| | | SEQ | | SEQ |
|---|---|---|---|---|
| | Primer | ID | Primer | ID |
| Gene | Sequence (Forward) | NO | Sequence (Reverse) | NO |
| Ki-67 | AAGCCCTCCAGCTCCTAG | 1 | TCCGAAGCACCACTTCTTCT | 2 |
| | TC | | | |
| Nestin | GTTGACCAACGCTGGCG | 3 | CGGGCTACCTCCTCCGTCGT | 4 |
| | GGA | | | |
| Beta3- | GGCCAAGGGTCACTACA | 5 | GCAGTCGCAGTTTTCACACT | 6 |
| Tubulin | CG | | C | |
| Pax6 | ACATCTGGCTCCATGTTG | 7 | GGCAGCATGCAGGAGTATGA | 8 |
| | GG | | | |
| Lmx1-a | TGCTGTTAAATGTGGGG | 9 | TCACCCTTCTGAAGCTGTCG | 10 |
| | GCT | | | |
| TH | GCACCTTCGCGCAGTTCT | 11 | CCCGAACTCCACCGTGAA | 12 |

### 4. Dopamine Dosage by HPLC

Dopamine production was analyzed by high-pressure liquid chromatography (HPLC) after extraction from dopaminergic neurospheres cultivated in immersion (3D-i) or in air-liquid culture interface (3D-ALi) in 0.1 N perchloric acid (HClO4). Cells were lysed in a small volume (250 μL) for 15 min at 4° C. with a vigorous vortexing every 5 min. Supernatant was recovered after centrifugation and used for dopamine dosage immediately or stored at −20° C. Catecholamines were measured by HPLC with electrochemical detection in coulometric mode. Separation of the analytes was achieved on a reversed-phase column, Symmetry C-18, 5 mm (4.6-150 mm²) (Waters Corporation), in isocratic mode at a flow rate of 1 mL/min. The coulometric detector parameters, Coulochem III (Thermo scientific), were for conditioning cell at a potential of +200 mV. For analytical cell, an electrode 1 at a potential +100 mV and electrode 2 at a potential of −100 mV were used.

### 5. Western Blot

Total proteins were extracted from the dopaminergic neurospheres at 1 month of differentiation by homogenization of the cells in RIPA buffer (lx) and incubation for one hour on ice. The lysate was centrifuged at 13 r.p.m. for 10 minutes and the supernatant was used for western blot analysis. For each condition, 10, 20, 30 and 40 μg of protein were analyzed by western blot to evaluate the expression of protein tyrosine hydroxylase.

### 6. Electrophysiology by MEA Array

A dissection microscope was used to transfer dopaminergic neurospheres to the center of a porous MEA device. Eight-four recording electrodes were selected for each given condition in triplicate. Then, an amplifier and data acquisition system for electrophysiological recordings was used. Measure the signal-to-noise ratio (SNR) as the standard deviation of the voltage during a 30 min recording, using the signal as the average peak-to-peak voltage of the spikes recorded in the same 30 min periods.

### 7. Neurospheres Plating on Extracellular Matrix, Immunocytochemistry and Electron Microscopy

At day 29, the neurospheres were plated on polyornithine/laminine-coated tissue culture plates in maturation neurobasal medium supplemented with 1% of penicillin/streptomycin, 1% Non-essential amino-acids, 1% B-27 supplement, 3 μM of CHIR99021, 0.5 mM of cAMP, 20 ng/mL of GDNF, 20 ng/mL of GDNF, 5 ng/mL of FGF20, 1 ng/mL of TGF-B3 and 1 μM of Compound E. After 1 week of culture on coverslips, cells on glass coverslips were fixed with 1 mL of 4% paraformaldehyde in PBS for 30 minutes at room temperature. Cells were then washed three times with 2 mL of PBS. Cells were permeabilized with PBS containing 0.3% Triton X-100 (Sigma) and incubated overnight with primary antibodies in PBS containing 5% bovine serum albumin (Sigma).

Cells were washed three times with 2 mL of PBS before incubation for 1.5 hour with the secondary antibody in PBS containing 5% bovine serum albumin. Cells were washed three times with PBS before exposure to DAPI 300 nM in PBS (Sigma) for 15 minutes at room temperature. After three washes in PBS, cells were rinsed with water and mounted in glass slides using Fluorsave reagent (Merck Millipore). Moreover, dopaminergic neurospheres plated on the glass coverslips were dehydrated, then covered with a gold nanolayer (20 nm) and visualized by electronic microscopy.

### B. Results

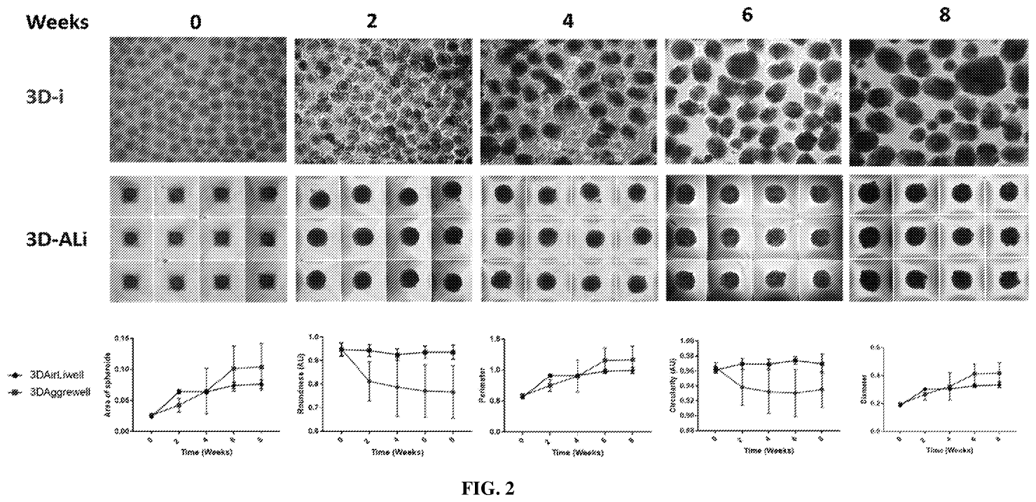

First, the homogeneity of dopaminergic neurospheres was studied over 60 days by using the dopaminergic specification protocol described previously. Dopaminergic neurospheres with the 3D conventional culture “3D-Immersion” was compared with 3D-AirLiwell neurospheres. As a practical reminder, in the standard method “3D-Immersion”, the spheroids are first forced aggregated in plastic microwells, then removed and transferred in standard culture plates maintained under agitation. In contrast, with the “3D-AirLiwell” method, the spheroids are forced to aggregation in non-adhesive microwells molded in medium-permeable agarose. In contrast to 3D-Immersion, they are individually maintained into the microwells without any transfer, immersion and agitation. A hemi-permeable membrane at the bottom of the molded microwells allows their long-term stability and also establish air/liquid interface conditions that favor gas exchanges with air. To compare and characterize more precisely the morphology of dopaminergic spheroids in 3D-Immersion versus 3D-AirLiwell, a dopaminergic differentiation during 2 months was performed and the evolution of their size and shape was followed by light microscopy. As shown in FIG. 2, there was no significant difference in both the round shape and size between the two methods on the week 0. However, after 2 weeks days of culture the dopaminergic neurospheres grown with 3D-AirLiwell were more homogeneous than those grown on 3D-Immersion were. A software-based quantification of the range of the distributions of the area, roundness, perimeter, circularity and diameter (FIG. 2), showed a clear lower variability with 3D-AirLiwell. In addition, the spheroids, which initially have a round shape, kept their round morphology with 3D-AirLiwell while those in 3D-Immersion evolved into more heterogeneous and irregular shapes.

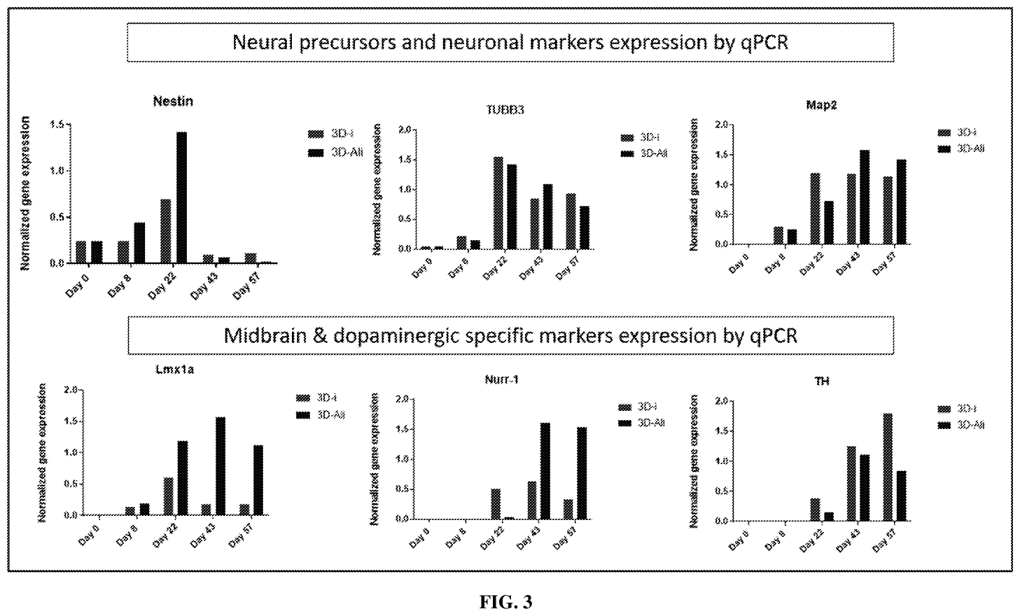

Then, the dopaminergic specification was monitored more quantitatively, at different time points, by using Q-RT-PCR. In addition to dopaminergic specification, the expression of some markers involved in early or late neural induction was analyzed, as well as cell proliferation at different time points during 2 months of differentiation (FIG. 3). First, no difference was observed between the two 3D cell cultures methods for the expression of Ki67, a cell proliferation marker. Nestin, which is also an early NPC marker and BetaIII-tubulin, a neuronal marker, did not show any significant regulation. These proteins are generally not up-regulated in cells during the neural induction process, but more reassembled in the cytosol to form more dense fibers of the cytoskeleton.

In contrast and expectedly, Pax-6, a transcription factor that is up-regulated during early neural induction, was increased in the “3D-AirLiwell” spheroids starting from day 15 until the end of the differentiation. Together, these observations reinforce the ability of 3D-AirLiwell to favor the switch between hESC and NPCs. Regarding markers of specification, Lmx1a, (which is an early midbrain marker up-regulated in dopaminergic precursors), was also up-regulated in 3D-AirLiwell spheroids from week 2 until the end of the differentiation. Finally, Tyrosine Hydroxylase, which is a marker of mature dopaminergic neurons, showed an up regulation in 3D-Immersion spheroid. Thus, These Q-RT-PCR results let suggest that the 3D-AirLiwell method allows a better midbrain regionalization of NPCs but clearly confirm a lack of maturation.

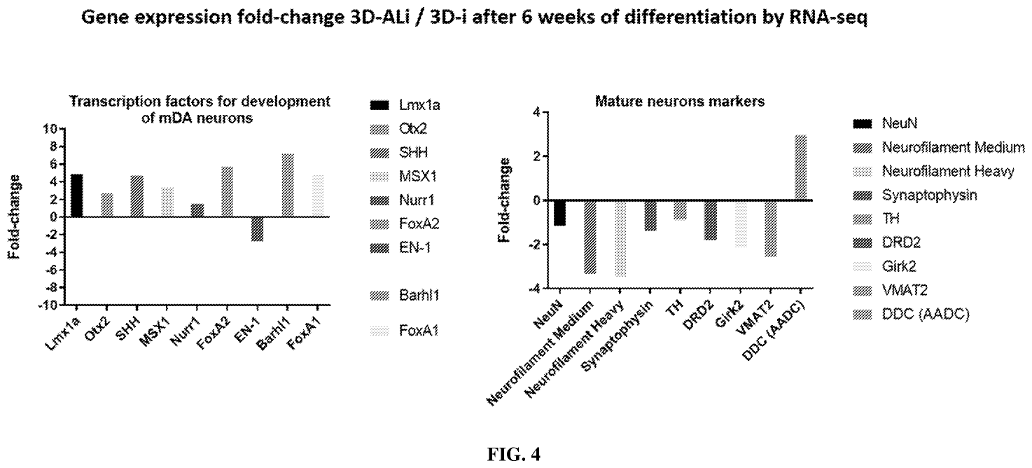

To have a global view about gene expression, an RNA-seq Analysis was performed to compare the spheroids cultivated by both methods at week 6 (=day 43) for 3 different cell batches of HS420 cell line. In the graphs of FIG. 4 the fold change between 3D-AirLiwell and 3D-immersion, positive bars mean up-regulation in 3D-AirLiwell and inversely negative bars mean up-regulation in 3D-Immersion. The combination of Lmx1a, Otx-2, FoxA2 is the best association to estimate the early regional status of NPCs, since dopaminergic NPCs are derived from midbrain ventral cells, i.e. triple-positive cells. On the left graph (FIG. 4), these 3 markers are up-regulated in spheroids grown in “3D-AirLiwell”. Moreover, others transcription factors (ex: Nurr-1, MSX1, FoxA2) involved in development of dopaminergic neurons were also up-regulated in 3D-ALi confirming the suggestion of a better dopaminergic regionalization and specification of the NPCs. On the contrary, the late dopaminergic markers such as TH, EN-1 (engrailed-1), DRD2 (dopamine receptor), VMAT2 (dopamine transporter) are up-regulated in spheroids grown with the standard 3D-Immersion method (FIG. 4). Moreover, the expression of other specific markers of maturation was also analyzed. A lack of maturation in spheroids cultured with the 3D-AirLiwell method was confirmed as shown by the up-regulation of all markers of maturation (NeuN, Neurofilament heavy . . . , FIG. 4).

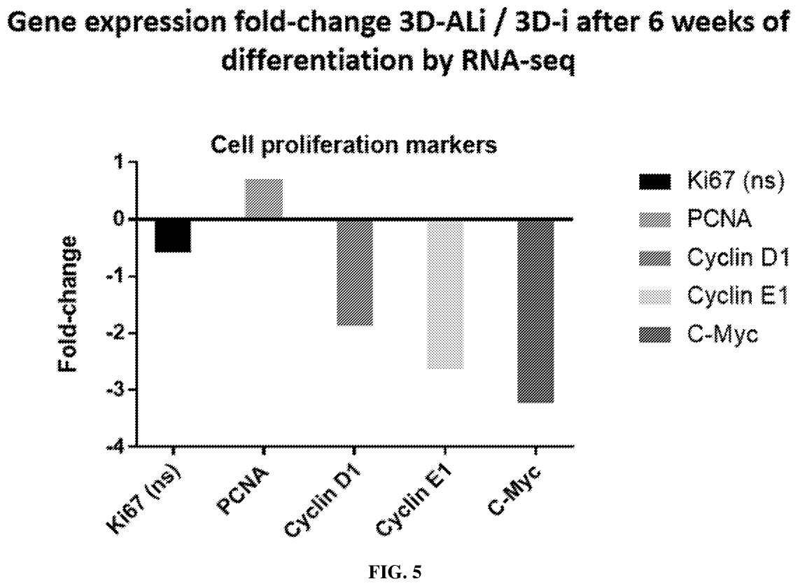

In addition, the expression of cell proliferation markers was also analyzed. As shown in the FIG. 5, the cell proliferation markers are all down-regulated in the neurospheres cultured by the 3D-ALi method, suggesting a great advantage in terms of tumor risk, which may be lower for cells cultured by this technique.

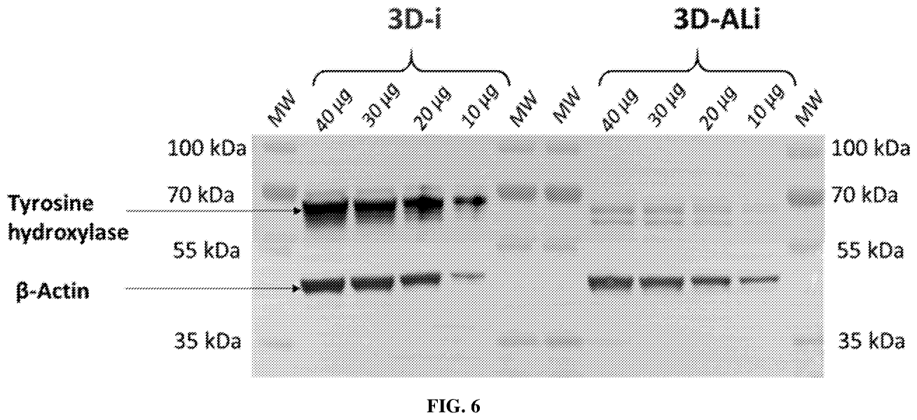

However, the lack of maturation of the dopaminergic neurospheres cultivated by the “3D-AirLiwell” was also confirmed at the protein level. A dopaminergic differentiation with both 3D methods “3D-Immersion” and “3D-AirLiwell” was performed and a western blot analysis of the spheroids was performed at day 33 of differentiation (FIG. 6). As expected, we observed a lower amount of Tyrosine hydroxylase protein in the samples from spheroids cultivated in “3D-AirLiwell”, suggesting a lack of maturation by this novel method.

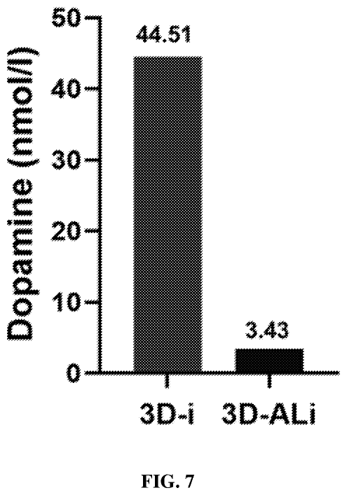

Furthermore, the dopamine production was analyzed by high-pressure liquid chromatography (HPLC) after extraction from dopaminergic neurospheres cultivated in immersion (3D-i) or in air-liquid culture interface (3D-Ali). As expected, the dopamine amount produced by the 3D-ALi dopaminergic neurospheres was lower than the 3D-i neurospheres (FIG. 7).

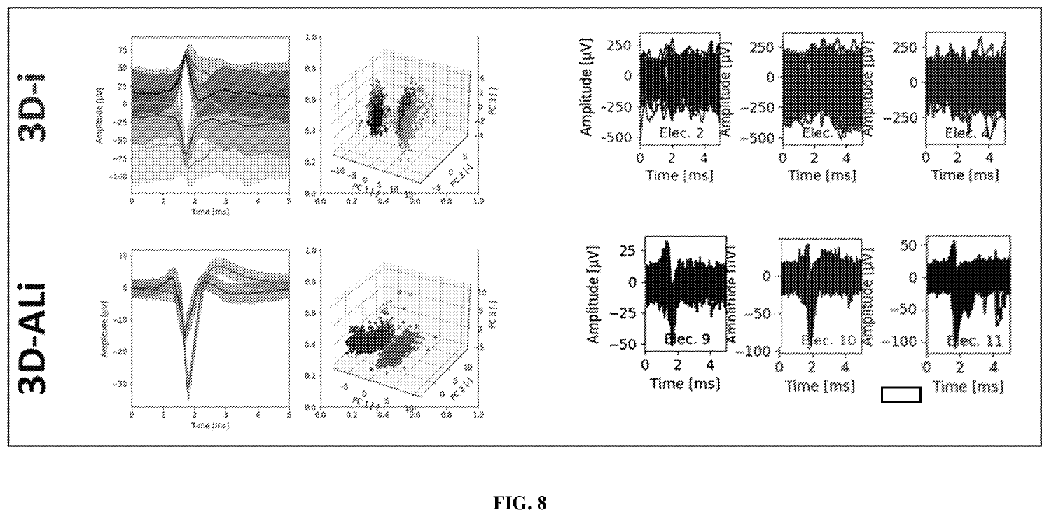

Concerning the electrophysiological aspect of the dopaminergic neurospheres, the spontaneous electrical activity generated by the neurons differentiated by the two techniques was measured. Both neurospheres cultivated in 3D-immersion and 3D-AirLiwell were alive and electro-physiologically active. As shown in the left part of the FIG. 8, the waveform of the 3D-AirLiwell neurospheres signal is more homogeneous and grouped into 2 similar shapes, while for 3D immersion, the shape is more heterogeneous and grouped into more than 4 different shapes. Dopaminergic neurospheres cultivated in 3D-immersion showed significant higher frequency, amplitude of spikes and burst and a higher number of spikes, number of bursts suggesting more neuronal network activity. However, neurospheres grown in 3D-AirLiwell show calm and stable activity over time while those in 3D-immersion have more epileptic activity (FIG. 8).

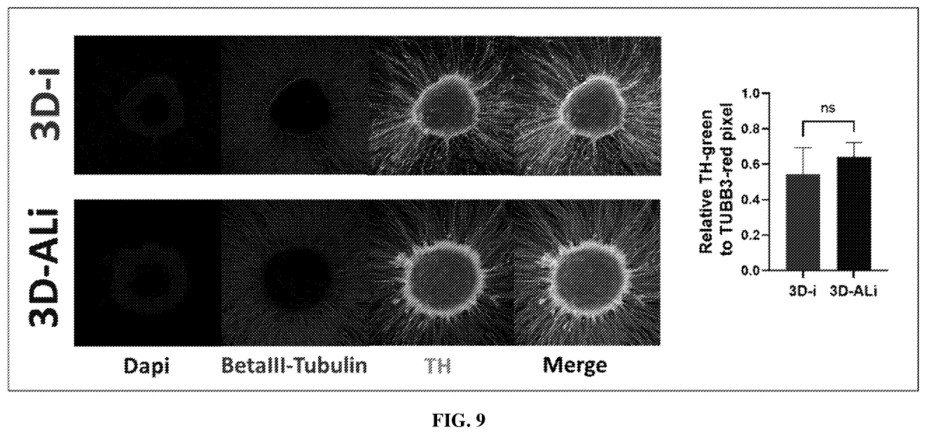

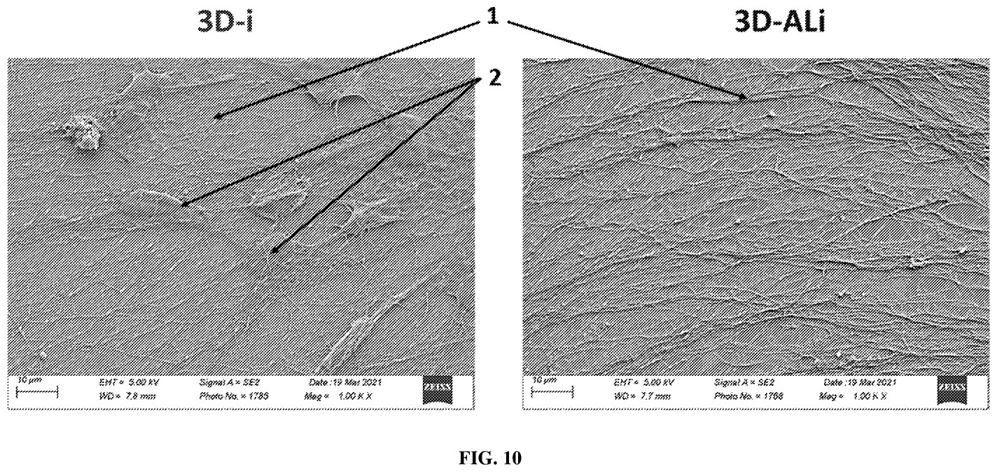

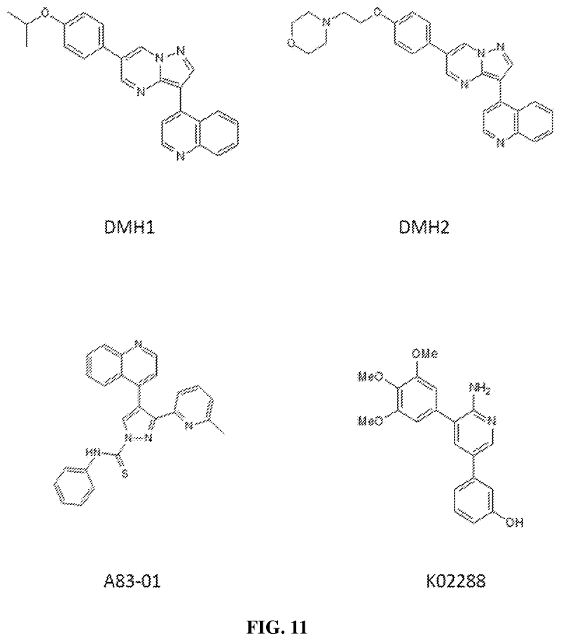

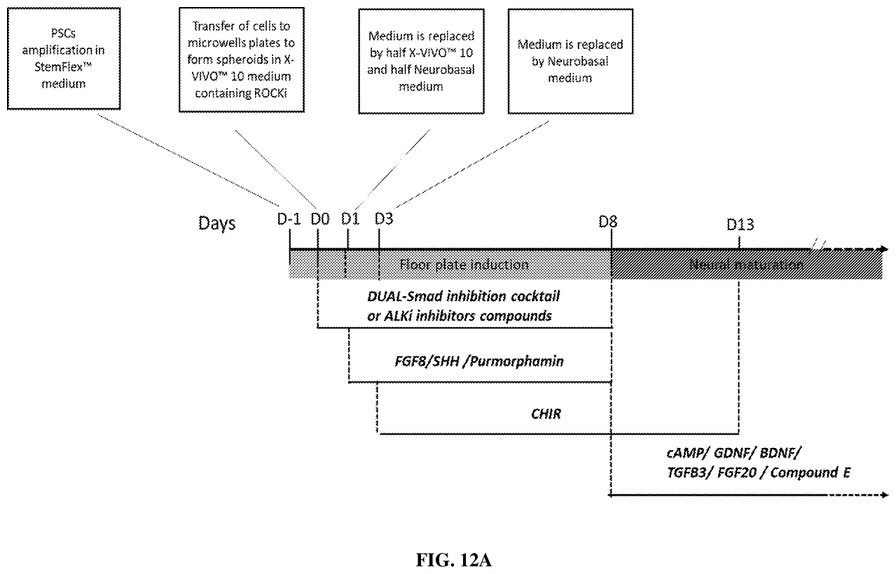

Neuronal maturation of dopaminergic neurospheres was induced by their plating on extracellular matrix-coated (ECM) plates at week 4 of differentiation. After 1 week of maturation, an immunofluorescence staining was performed with the neuronal marker beta-III tubulin and tyrosine hydroxylase (TH), a specific marker of dopaminergic neurons (FIG. 9). Abundant neurons were obtained around plated spheres with both methods, confirming that 3D-AirLiwell spheroids kept the neuronal maturation properties. The mature neurons on ECM-coated plates were also observed by electron microscopy (FIG. 10). This allowed to analyze the neuronal network and the shape of the cells with a better resolution. With the immersion method, the presence of invading large flattened cells (2) was observed (through DAPI staining and electron microscopy) underlying the neuronal network (1), suspected to be proliferating NPC (because shown to be Nestin+, Pax-6+, data not shown). In contrast and clearly, 3D-AirLiwell did not induced this cell population, showing a higher level of homogeneity of the cell preparation.

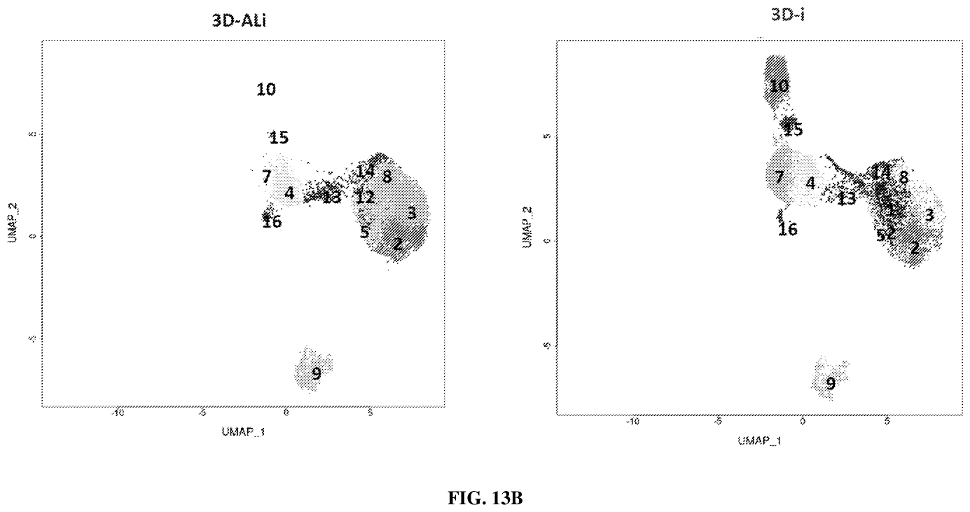

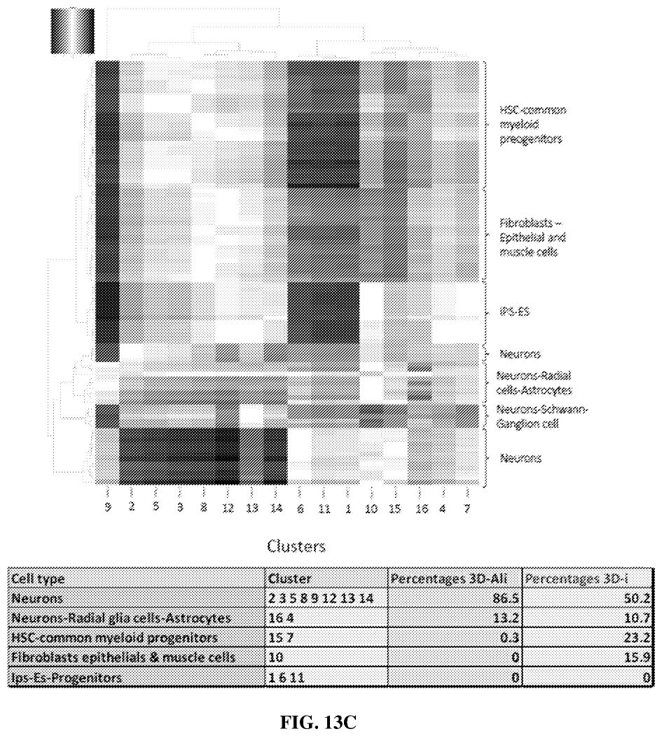

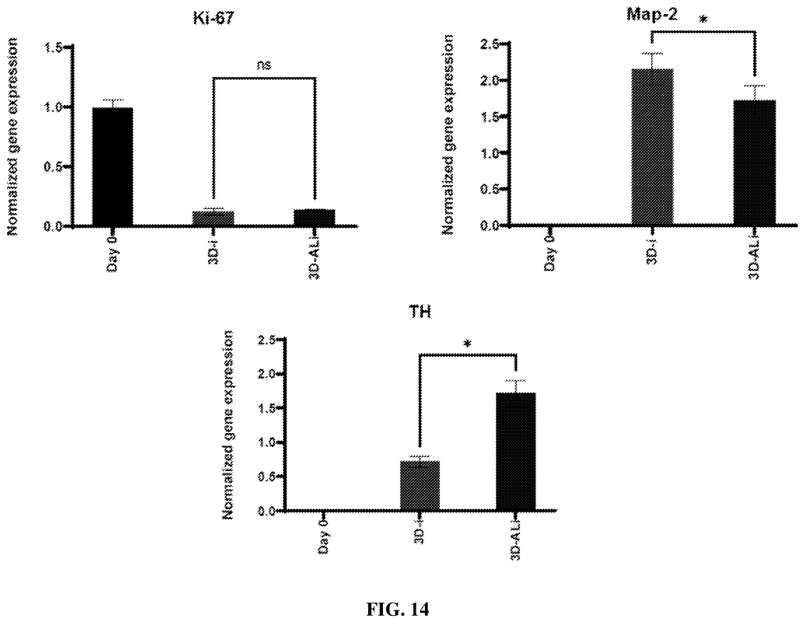

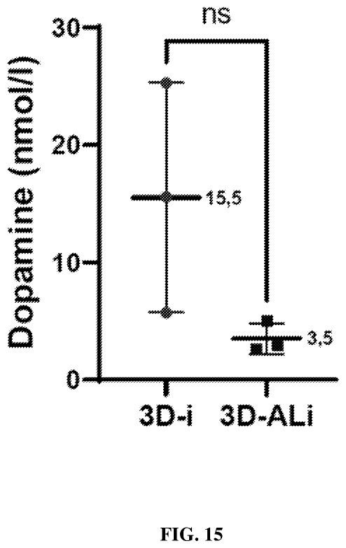

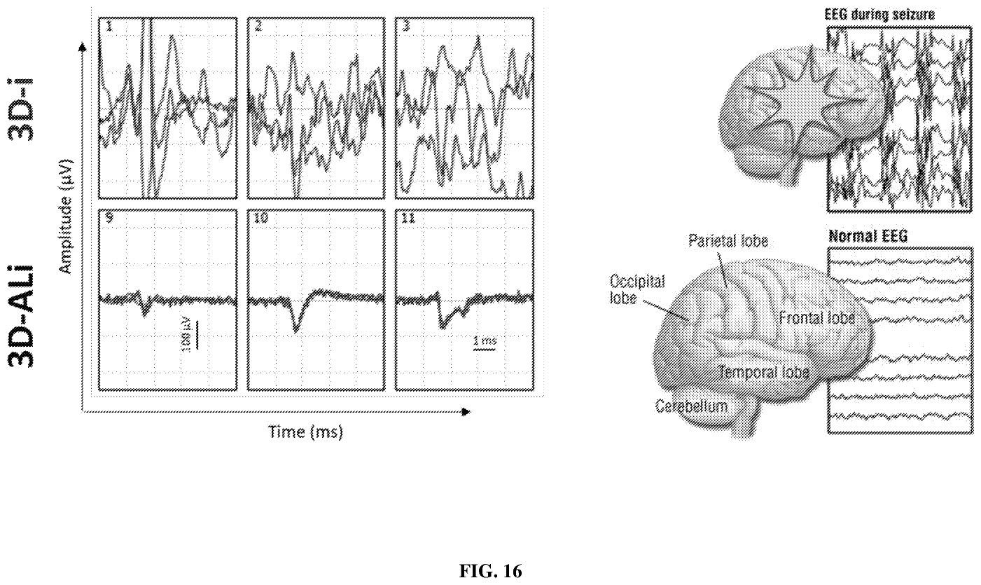

To further characterize the neurospheres produced by the 3D-Ali method and the 3D-i method, single cell RNA sequencing was performed to determine the phenotype of the different cells in the neurospheres (FIG. 13A). The majority of cells in the 3D-Ali neurospheres were in neuron clusters (86.5%), whereas only half of the cells in the 3D-i neurospheres were in neuron cell type clusters. The 3D-I neurospheres had a higher percentage of myeloid progenitors (23.2% in 3D-i vs. 0.3% in 3D-Ali) and fibroblasts (15.9% in 3D-i vs. 0% in 3D-Ali) (FIG. 13B-C). Thus, 3D dopaminergic differentiation under 3D-Ali condition yields more homogenous cell populations than under 3D-I conditions. It was also found that tyrosinehydroxylase(TH) mRNA increased under 3D-Ali conditions (FIG. 14), but no difference in dopamine content was detected (FIG. 15. The 3D-Ali conditions also provided for electrical signals that were more indicative of stable neurons (FIG. 16).

To improve the maturation step, 2.5% or 10% of Laminin 511 (Takkara) or Geltrex® (A growth factors reduced extracellular matrix, ThermoFisher) will be added into the cell culture medium to promote neuronal maturation of spheroids starting from day 1 of differentiation to the end and from day 13 to the end. RGD peptides could also be tested to enhance maturation, by adding them to the culture medium similarly to laminin or geltrex.

The second strategy will consist in the culture of dopaminergic spheroids under hypoxic conditions starting from day 1 of differentiation to the end and from day 13 to the end. In addition, the combination of hypoxia and the addition of extracellular matrix (Laminin or Geltrex) or RGD peptides in the medium will also be tested. The combination of these will be applied from day 1 of differentiation to the end and from day 13 to the end.

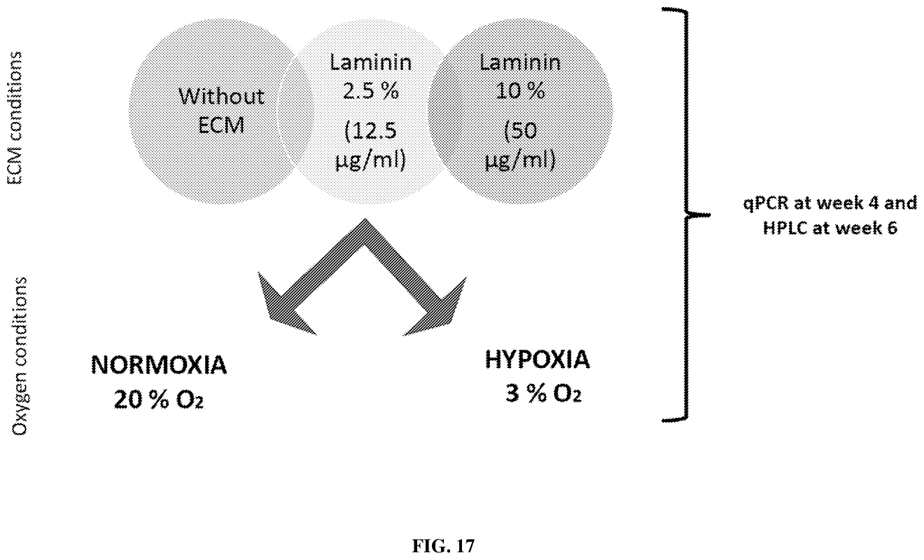

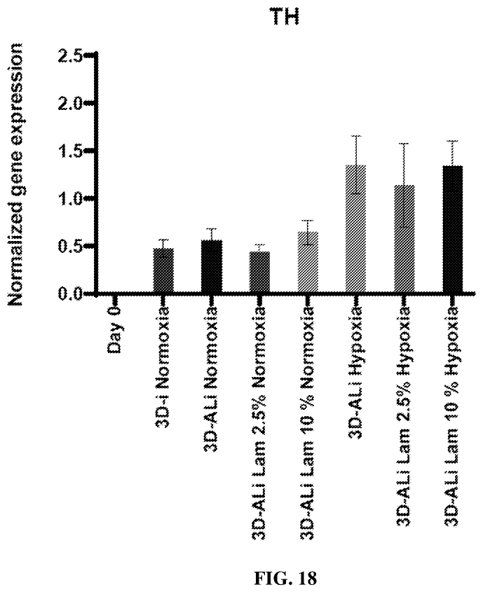

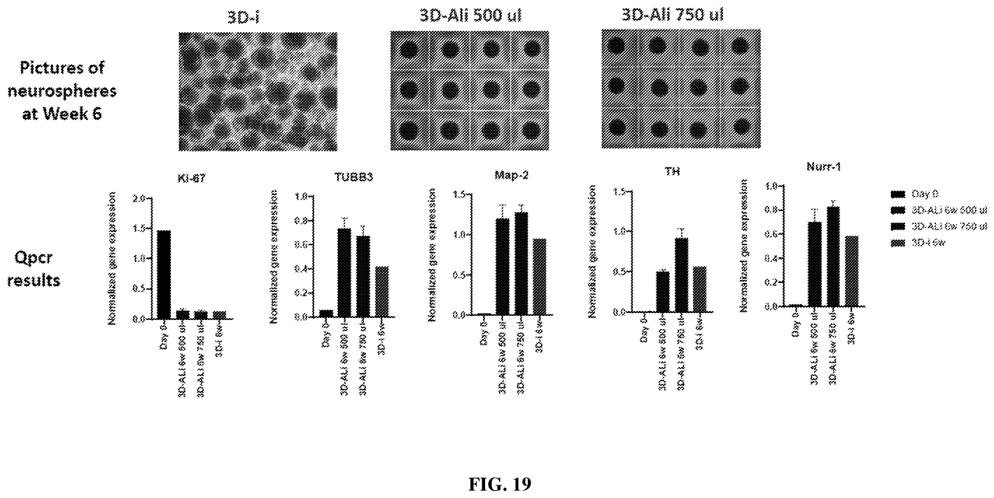

The inventors tested whether the additional of an extracellular matrix and/or hypoxia could improve the development of mature neurons. 2.5% and 10% laminin and normoxia (20% oxygen) or hypoxia (3% oxygen) was tested (FIG. 17). As shown in FIG. 18, hypoxia appeared to increase and accelerate the maturation of dopaminergic neurons at day 29, and laminin did not provide any apparent effect either in normoxic or hypoxic conditions. It is further hypothesized that the effect demonstrated by hypoxia may be recapitulated with the addition of HIF-1alpha stabilizers to the cell culture medium. It was further hypothesized that other changes to the cell culture, such as modification of the volume of the cell culture medium could provide for an improvement in the maturation of neurons. The inventors found that the ratio between the number of neurospheres and the volume of the cell culture medium can provide for an improvement in the maturation of the cell. In AirLiwell (6 well plate), a seeding 1.5 million cells per well (6 well AirLiwell plate) yields 2350 neurospheres. Under these conditions, 750 μl of cell culture medium provides for a more effective method for differentiating the neurons (FIG. 19).

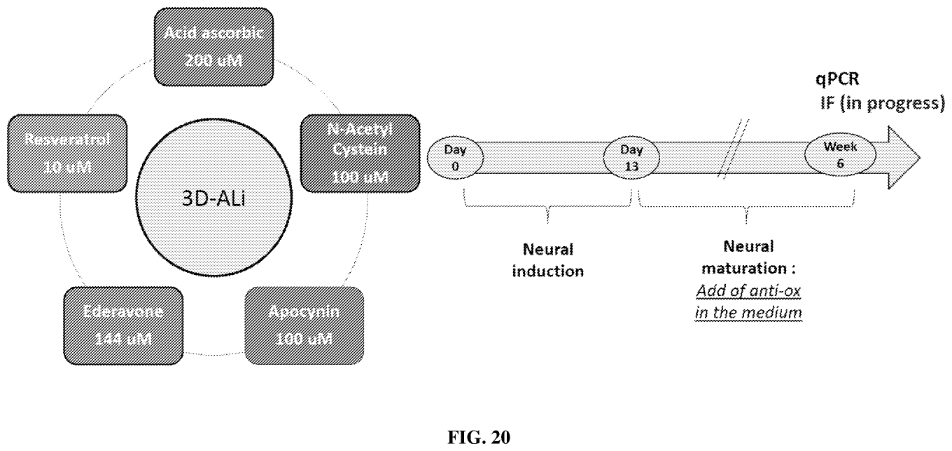

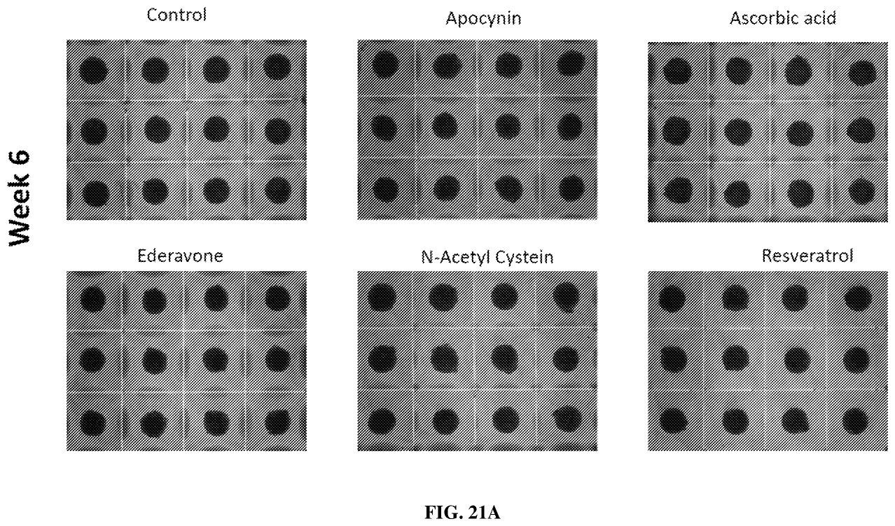

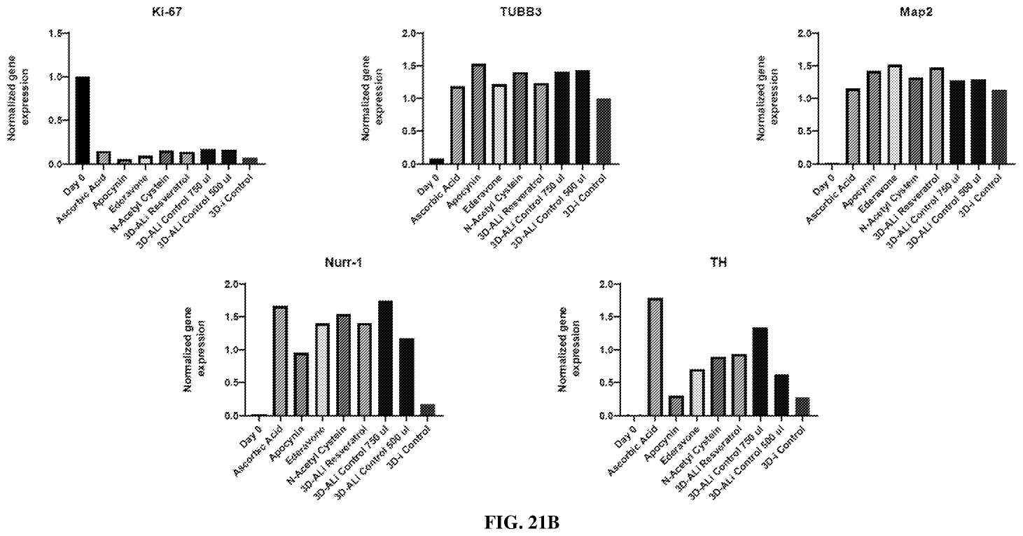

The inventors also tested the effects of different antioxidants on three-dimensional dopaminergic neuron differentiation. FIG. 20 shows the protocol design with the addition of anti-oxidants. As shown in FIGS. 21A-B, the anti-oxidants did not have an impact on global neuronal differentiation. Comparable gene expression for the neuronal markers b3-tubulin (TUBB3) and MAP2 was seen, and comparable down-regulation of the proliferation marker, ki67 was also seen. Most of the tested anti-oxidants diminish TH expression during dopaminergic neuron differentiation. However, it was found that ascorbic acid (Vitamin C) lead to a marked enhancement in DA neuron differentiation (FIG. 21B).

It is also contemplated that an overexpression of factors promoting dopaminergic neuronal maturation can be stably transduce within hESC (HS420 cell line) to improve dopaminergic maturation. Selected factors may include Nurr-1 and Pitx3. Indeed, these two transcription factors are very important in vivo for the maturation and maintenance of dopaminergic neurons. Reinforcing this choice, these 2 transcription factors were weakly expressed by the spheroids. Also, numerous studies highlight that Nurr1 and Pitx3 are critical for the maturation of dopaminergic neurons. Of note, a neuronal-specific promoter will be used to overexpress these 2 transcription factors. As example, the Tα1 α-tubulin promoter validated in a previous study will be tested to specifically target these factors in neurons. [Suter D M, Cartier L, Bettiol E, Tirefort D, Jaconi M E, Dubois-Dauphin M, Krause K H Stem Cells. 2006 Mar. 24(3):615-23. doi: 10.1634/stemcells.2005-0226. Epub 2005 Nov. 17. PMID: 16293575].

### Example 2 Neuronal Cell Differentiation in 3D Cell Culture

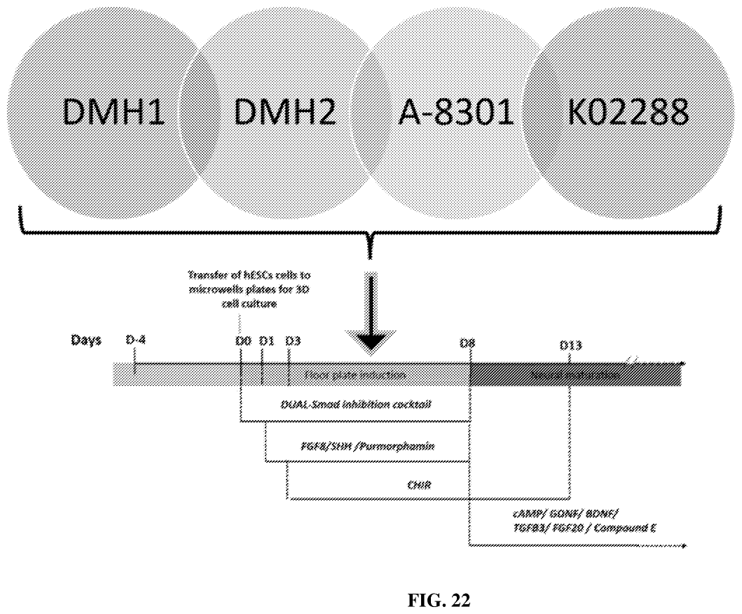

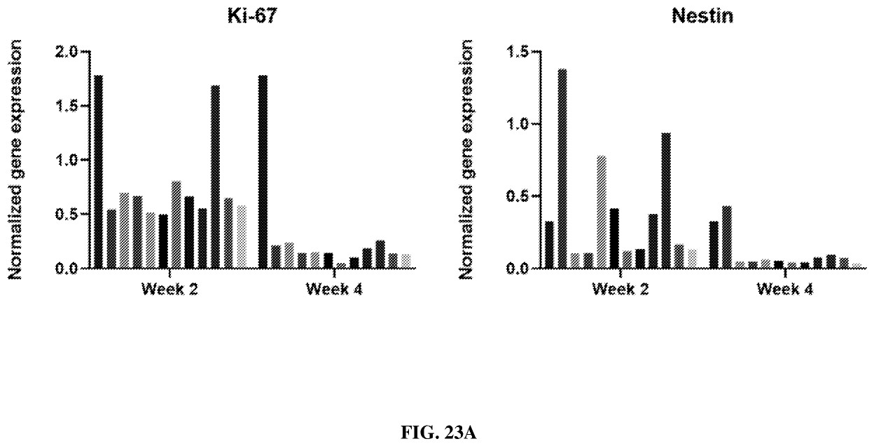

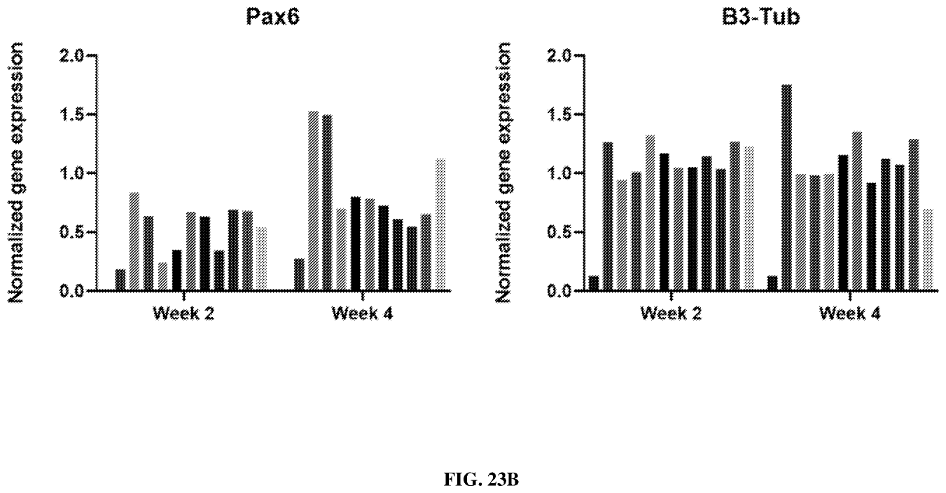

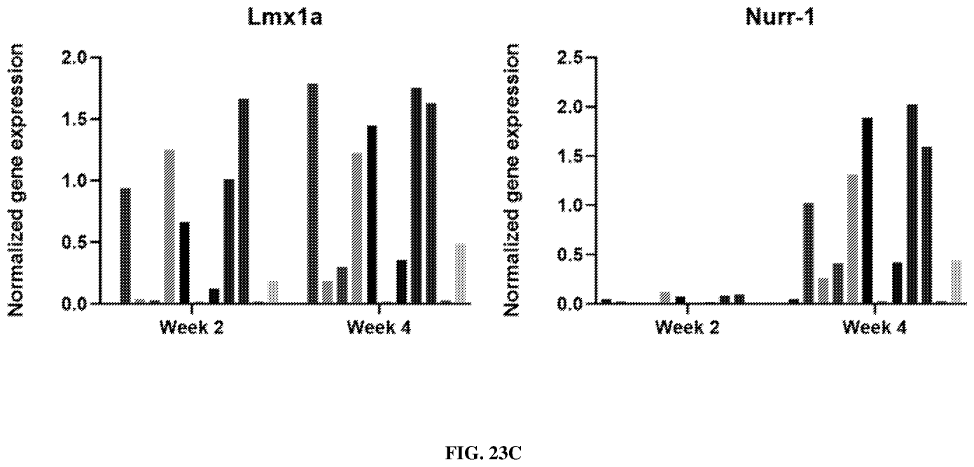

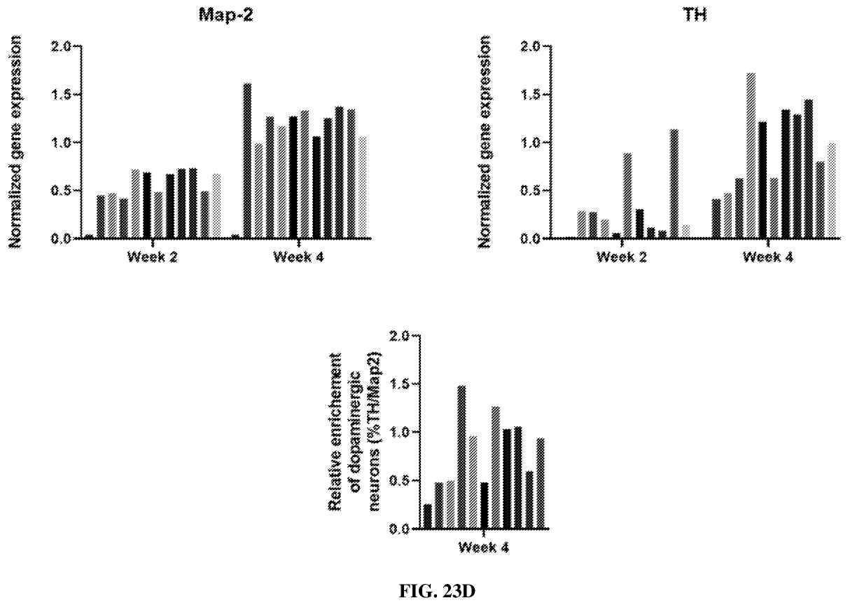

To study the effects of others ALK inhibitors (ALKi) described previously and to possibly replace the dual-SMAD inhibition in the protocol using the 3D-AirLiwell technology, human embryonic stem cell line HS420 was differentiated into dopaminergic neurospheres as described previously in Example 1, and the effect on hESC neural induction of DMH1, DMH2, K02288 and A8301 alone or combination was compared to the combination of LDN193189 and SB431542 compounds. The results of differentiation were analyzed by q-PCR after 2 and 4 weeks of dopaminergic differentiation (FIG. 23A-D). Cells were exposed to the compounds at day 0 during one week, at the tested concentrations of 0.2 M and 0.8 M in a minimal medium suited for early neural induction. In addition to ALKi compounds, the compounds and proteins mentioned in the protocol and used for the first week of induction as described above will be added to the culture medium such as purmorphamine, SHH and FGF-8 (FIG. 22). It was found that DMH1, DMH2, A-8301, and K02288 have an activity comparable to LDN/SB (dual smad inhibition) with respect to general neural differentiation. In contrast, the different ALK inhibitors have differential effects on dopaminergic differentiation: DMH1 and DMH2 roughly equipotent as compared to LDN/SB and A-8301 and K02288 have no major effects on dopaminergic differentiation. The combination of DMH2 and A-8301 provided for a synergistic effect in a 2-dimensional cell culture.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for differentiating stem or progenitor cells into neural cells, the method comprising (i) contacting the stem or progenitor cells with a differentiation composition, wherein the differentiation composition comprises one or more of the ALK inhibitors: DMH1, DMH2, K02288, and A83-01; and (ii) culturing the cells in microwells to form spheroids and/or neurospheres; wherein the ALK inhibitor excludes LDN193189 and SB431542; wherein the cells, spheroids, and/or neurospheresare are cultured under hypoxic conditions; and wherein the ratio of the number of spheroids or neurospheres to the volume of cell culture media is about 1000 spheroids or neurospheres to 200-500 μl cell culture media.

2. A method for differentiating stem or progenitor cells into neural cells, the method comprising (i) contacting the stem or progenitor cells with a differentiation composition; and (ii) culturing the cells in microwells to form spheroids and/or neurospheres.

3. A method for differentiating stem or progenitor cells into neural cells, the method comprising (i) contacting the stem or progenitor cells with a differentiation composition, wherein the differentiation composition comprises one or more of the ALK inhibitors: DMH1, DMH2, K02288, and A83-01; and (ii) culturing the cells in microwells to form spheroids and/or neurospheres.

4. The method of claim 3, wherein the ALK inhibitor consists of DMH1, DMH2, K02288, or A83-01.

5. The method of claim 3 or 4, wherein the ALK inhibitor comprises or consists of DMH2.

6. The method of claim 3, wherein the ALK inhibitors consist of DMH1 and DMH2.

7. The method of claim 3, wherein the ALK inhibitors consist of K02288 and DMH2.

8. The method of claim 3, wherein the ALK inhibitors consist of A8301 and DMH2.

9. The method of any one of claim 8, wherein the ALK inhibitors exclude LDN193189 and/or SB431542.

10. The method of any one of claims 3-9, wherein the concentration of ALK inhibitor in the differentiation composition is 0.1-1 μM.

11. The method of any one of claims 2-10, wherein the stem or progenitor cells comprise induced pluripotent stem cells (iPSCs) or embryonic stem (ES) cells.

12. The method of claim 11, wherein the stem or progenitor cells comprise embryonic stem (ES) cells.

13. The method of claim 12, wherein the ES cells are human ES cells.

14. The method of claim 13, wherein the human ES cells comprise HS420 cells.

15. The method of any one of claims 2-8, wherein the stem or progenitor cells comprise totipotent, pluripotent, or multipotent stem cells.

16. The method of any one of claims 3-13, wherein the contacting the cells comprises contacting the cells for a time period of about 1-7 days of substantially continuous contact.

17. The method of any one of claims 3-16, wherein the cells are contacted with 0.01-5 μM of the ALK inhibitor.

18. The method of claim 17, wherein the cells are contacted with 10 μM ALK inhibitor.

19. The method of any one of claims 2-18, wherein the method further comprises contacting the stem or progenitor cells with a Rho Kinase (ROCK) inhibitor.

20. The method of claim 19, wherein the ROCK inhibitor comprises Y27632.

21. The method of claim 19 or 20, wherein the cells are contacted with 5-15 μM ROCK inhibitor.

22. The method of any one of claims 2-21, wherein the cells are contacted with the ROCK inhibitor prior to contact with the differentiation composition.

23. The method of any one of claims 2-21, wherein the cells are contacted with the ROCK inhibitor for a period of time that overlaps with the contact with the differentiation composition.

24. The method of claim 22 or 23, wherein the cells are contacted with the ROCK inhibitor for a time period of 1-48 hours.

25. The method of any one of claims 2-24, wherein the method excludes contacting the cells with a Smad inhibitor and/or a BMP4 inhibitor.

26. The method of claim 25, wherein the method excludes contacting the cells with LDN193189 and/or SB431542.

27. The method of any one of claims 2-26, wherein the method excludes dual or mono-Smad inhibition.

28. The method of any one of claims 2-27, wherein the method excludes contacting the cells with a Noggin protein.

29. The method of any one of claims 2-28, wherein the neural cells are further defined as dopaminergic neurons, glutamatergic, serotoninergic, cholinergic, GABAergic, motoneurons, astrocytes, or oligodendrocytes.

30. The method of claim 29, wherein the neural cells are defined as dopaminergic neurons.

31. The method of any one of claims 2-30, wherein contacting the cells with a compound, ALK inhibitor, or composition comprises culturing the cells in a cell culture medium comprising the compound, ALK inhibitor, or composition.

32. The method of claim 31, wherein the cell culture medium comprises one or more of DMEM medium, Neurobasal medium, a GSK inhibitor, cAMP, GDNF, BDNF, amino acids, X-VIVO medium, antimicrobial agents, B-27 supplement, N-2 supplement and L-glutamin, sonic hedgehog protein (SHH), purmorphamin, FGF-8 protein (fibroblast growth factor 8), FGF-20, TGF-B3, and a gamma secretase inhibitor.

33. The method of any one of claims 24-32, wherein the cells are further contacted with a GSK3 (glycogen synthase kinase 3) inhibitor for a period of time.

34. The method of claim 33, wherein the GSK3 inhibitor comprises CHIR99021.

35. The method of claim 33 or 34, wherein the GSK3 inhibitor was added after 3 days after contact with the differentiation medium.

36. The method of any one of claims 33-35, wherein the time period is 5-15 days.

37. The method of claim 36, wherein the time period is 10 days.

38. The method of any one of claims 32-37, wherein the method comprises or further comprises contacting the cells with one or more of FHF8, SHH, and purmorphamin for a period of time.

39. The method of claim 38, wherein the cells are contacted with FHF8, SHH, and/or purmorphamin one day after contact with the differentiation medium.

40. The method of claim 38 or 39, wherein the period of time is 3-10 days.

41. The method of claim 40, wherein the period of time is 7 days.

42. The method of any one of claims 32-41, wherein the method comprises or further comprises contacting the cells with one or more of cAMP, GDNF, BDNF, TGFB3, FGF20, and a gamma secretase inhibitor for a period of time.

43. The method of claim 42, wherein the cells are contacted with cAMP, GDNF, BDNF, TGFB3, FGF20, and/or a gamma secretase inhibitor eight days after contact with the differentiation medium.

44. The method of claim 42 or 43, wherein the period of time is 15-40 days.

45. The method of any one of claim 2-44, wherein the method comprises or further comprises contacting the cells with ascorbic acid.

46. The method of claim 45, wherein the ascorbic acid in contact with the cells is at a concentration of 100-400 μM.

47. The method of claim 46, wherein the ascorbic acid in contact with the cells is at a concentration of 200 μM.

48. The method of any one of claims 45-47, wherein the cells are contacted with ascorbic acid at a period of time of 8-15 days after contact with the differentiation medium.

49. The method of claim 48, wherein the cells are contacted with ascorbic acid at a period of time of 13 days after contact with the differentiation medium.

50. The method of any one of claims 2-49, wherein the cells are contacted with ascorbic acid for a time period of 10-40 days.

51. The method of any one of claims 2-50, wherein the differentiation composition comprises or the method further comprises contacting the cells, spheroids, or neurospheres with an extracellular matrix for a period of time.

52. The method of claim 51, wherein the extracellular matrix comprises Laminin and/or Geltrex.

53. The method of any one of claims 2-44, wherein the differentiation composition excludes or the method excludes contacting the cells, spheroids, or neurospheres with an extracellular matrix.

54. The method of any one of claims 2-53, wherein the differentiation composition comprises or the method further comprises contacting the cells, spheroids, or neurospheres with RGD peptides for a period of time.

55. The method of any one of claims 2-54, wherein the cells, spheroids, and/or neurospheresare are cultured under hypoxic conditions for a period of time.

56. The method of any one of claims 42-55, wherein the period of time is from 1 to 60 days.

57. The method of claim 55 or 56, wherein the hypoxic conditions comprise 0-10% oxygen.

58. The method of claim 57, wherein the hypoxic conditions comprise 3% oxygen.

59. The method of any one of claims 2-58, wherein the method further comprises contacting the cells with a HIF-1α stabilizer.

60. The method of claim 59, wherein the HIF-1α stabilizer comprises one or more of dimethyloxalyl glycine, FG4592, CoCl₂, Deferoxamine mesylate, cyclometalated iridium(III) metal complex 1a, 1-(Imidazol-1-ylmethyl)-3,5-diphenylpyrazole, 3,5-Diphenyl-1-(pyrazole-1-ylmethyl) pyrazole, N-[(3,5-diphenylpyrazol-1-yl) methyl]-N-phenylaniline, (3,5-Diphenylpyrazol-1-yl) methyl]diethylamine, and (3,5-Diphenylpyrazol-1-yl) methyl] diisopropylamine.

61. The method of any one of claims 2-60 wherein the ratio of the number of spheroids or neurospheres to the volume of cell culture media is about 1000 spheroids or neurospheres to 200-1000 μl cell culture media.

62. The method of any one of claims 2-60 wherein the ratio of the number of spheroids or neurospheres to the volume of cell culture media is about 1000 spheroids or neurospheres to 200-500 μl cell culture media.

63. The method of claim 61 or 62, wherein the ratio is maintained for at least a time period of Day 0 to Day 42, wherein Day 0 is the day that the cells are first contacted with the differentiation medium.

64. The method of any one of claims 3-63, wherein the stem or progenitor cells comprise exogenously expressed Nurr-1 and/or Pitx3.

65. The method of any one of claims 2-64, wherein the stem or progenitor cells comprise a heterologous nucleic acid encoding for a Nurr-1 and/or Pitx3 protein or a functional fragment thereof.

66. The method of any one of claims 2-65, wherein the neural cells are further defined as Nestin+, Pax-6+, and Sox-1+ cells.

67. The method of any one of claims 2-66, wherein culturing the cells in microwells comprises culturing the cells on a substrate material comprising a hydrophilic and porous material layer with a plurality of lasting well-shaped indents on its outer surface supported by a semipermeable membrane on its inner surface, wherein said well-shaped indents have an aperture from 100 pm² to about 1 mm² and a bottom surface from about 100 pm² to about 1 mm², wherein said hydrophilic and porous material layer has a porosity which allows the passage of oxygen and cell nutrients.

68. The method of claim 67, wherein the semipermeable membrane has pores having a diameter from about 1 nm to about 200 μm, for example from about 2 nm to about 40 pm.

69. A spheroid or neurosphere produced by the method of any one of claims 3-68.

70. A population of cells, spheroids, or neurospheres produced by the method of any one of claims 3-68.

71. The population of cells, spheroids, or neurospheres according to claim 70, wherein the percentage of non-neural cells in the cell culture or spheroid after contact with the differentiation composition for a period of time is less than 30%.

72. The population of cells according to claim 71, wherein the period of time is 4-8 days.

73. A method of treating a disease in a mammalian subject comprising administering to the subject a therapeutically effective amount of the population of neural cells, spheroids, or neurospheres of claim 70.

74. The method of claim 73, wherein the disease comprises a neurodegenerative disease.

75. The method of claim 73 or 74, wherein the subject is a human subject.

76. The method of any one of claims 73-75, wherein the spheroids are dissociated prior to administration.

77. The method of any one of claims 73-75, wherein the method excludes dissociation of the spheroids prior to administration.
